(19) United States
(12) Patent Application Publication
(10) Pub. No.: US 2023/0258626 A1
(43) Pub. Date: Aug. 17, 2023

(54) FOLLICULAR HELPER T CELL PROFILE FOR IDENTIFYING PATIENTS WITH TYPE 1 DIABETES SUITABLE FOR TREATMENT WITH CTLA-4-IG

(71) Applicant: UCL BUSINESS LTD, London (GB)

(72) Inventors: Lucy Walker, London (GB); Niclas Thomas, London (GB)

(21) Appl. No.: 18/011,037

(22) PCT Filed: Jun. 16, 2021
(86) PCT No.: PCT/GB2021/051521

§ 371 Date: Dec. 16, 2022

(30) Foreign Application Priority Data
Jun. 16, 2020 (GB) 2009148.4

(51) Int. Cl. G01N 33/50

(52) U.S. Cl. CPC G01N 33/505; G01N 2800/042; G01N 2800/52

(57) ABSTRACT

Abstract: The present invention provides a method for identifying a subject with an autoimmune or inflammatory disease who is suitable for treatment with costimulation blockade therapy, the method comprising determining the profile of B helper T cells in a sample from the subject.

### FIELD OF THE INVENTION

The present invention relates to methods for identifying a subject who is suitable for treatment with costimulation blockade therapy and for predicting or determining whether a subject will respond to such treatment. Further, the invention relates to methods of treating or preventing an autoimmune or inflammatory disease in a subject. In particular, the invention relates to the use of the subject’s B helper T cell profile as a stratification tool, permitting the identification of individuals most likely to benefit from costimulation blockade.

### BACKGROUND TO THE INVENTION

Current nonautoantigen-specific treatments for autoimmune diseases and inflammatory diseases (e.g. Type I diabetes and rheumatoid arthritis) include therapies to reduce inflammation or to reduce the activity of the immune response. Such therapies include antiinflammatory drugs (e.g. anti-TNFα drugs like etanercept), and immunosuppressant therapies (e.g. anti-CD3 antibodies and corticosteroids like prednisone).

In general, the targets of many of these therapies, such as immunosuppressants, are ubiquitous and non-specific. By contrast, costimulation blockade therapies provide selective targets for the treatment of autoimmune and inflammatory conditions and are of major interest in autoimmune and inflammatory diseases. Costimulation blockade therapies are directed to decreasing T cell activation by inhibiting costimulatory signalling via a costimulatory molecule. The natural regulator of the CD28 costimulatory molecule is the inhibitory receptor CTLA-4, and a soluble version of this molecule has been developed for therapeutic use. Soluble CTLA-4 (a fusion protein with human immunoglobulin; CTLA-4-lg) is widely used in autoimmune diseases including rheumatoid arthritis (RA), psoriatic arthritis and juvenile idiopathic arthritis.

A randomised double-blind placebo controlled trial in individuals with new onset T1D demonstrated some efficacy of one such soluble CTLA-4-Ig, Abatacept (Orenica; Bristol-Myers Squibb), at 2 years compared with placebo (Orban, T., et al. (2011) Lancet, 378: 412-419). Although the beneficial effects were largely maintained a year following therapy cessation (Orban, T., et al. (2014) Diabetes Care, 37: 1069-1075), it was clear that some individuals benefited more than others, i.e. there was a high degree of heterogeneity in the response.

Thus, whilst costimulation blockade agents are proving to be a useful tool in the treatment of autoimmune and inflammatory diseases, not all patients respond to such treatments. Heterogeneity in the response to costimulation blockade drugs like Abatacept limits their utility as first line therapies, and therefore the ability to predict response to these reagents would have significant impact on how they are deployed in a clinical setting.

The present invention facilitates improved identification of patients who will respond to costimulation blockade therapy.

### SUMMARY OF THE INVENTION

B helper T cells, in particular follicular helper T cells (Tfh), are implicated in type 1 diabetes (T1D) and their development has been linked to CD28 costimulation. The present inventors tested whether Tfh were decreased by costimulation blockade (CTLA-4-Ig/Abatacept) in a mouse model of diabetes and in individuals with new onset T1D. Unbiased bioinformatic analysis confirmed changes in Tfh and revealed novel markers of costimulation blockade. Unexpectedly, the present inventors were able to use pre-treatment Tfh profiles to derive a model that could predict clinical response to costimulation blockade (CTLA-4-Ig/Abatacept). B helper T cell profiles, and in particular Tfh analysis, therefore represent a new stratification tool, permitting the identification of individuals most likely to benefit from costimulation blockade.

In one aspect, the present invention provides a method for identifying a subject with an autoimmune or inflammatory disease who is suitable for treatment with costimulation blockade therapy, the method comprising determining the profile of B helper T cells in a sample from the subject.

In a further aspect, the present invention provides a method for predicting or determining whether a subject with an autoimmune or inflammatory disease will respond to treatment with costimulation blockade therapy, the method comprising determining the profile of B helper T cells in a sample from the subject.

In a further aspect, the present invention provides a method of treating or preventing an autoimmune or inflammatory disease in a subject which comprises treating a subject with or at risk of developing an autoimmune or inflammatory disease with costimulation blockade therapy, wherein the subject has been identified as suitable for treatment with the costimulation blockade therapy by determining the profile of B helper T cells in a sample from the subject.

In a further aspect, the present invention provides a method of treating or preventing an autoimmune or inflammatory disease in a subject, wherein the method comprises the following steps:
(a) identifying or determining a subject with or at risk of developing an autoimmune disease who is suitable for treatment with costimulation blockade therapy by the method according to the invention; and
(b) treating the subject with costimulation blockade therapy.

In a further aspect, the present invention provides a method of treating or preventing an autoimmune or inflammatory disease in a subject which comprises treating a subject with or at risk of developing an autoimmune or inflammatory disease with costimulation blockade therapy, which subject has been identified or determined as suitable for treatment with costimulation blockade therapy by the method according to the invention.

In a further aspect, the present invention provides a costimulation blockade therapy for use in a method of treatment or prevention of an autoimmune or inflammatory disease in a subject, the method comprising:
(a) identifying or determining a subject with or at risk of developing an autoimmune or inflammatory disease who is suitable for treatment with costimulation blockade therapy by the method according to the invention; and
(b) treating the subject with costimulation blockade therapy.

In a further aspect, the present invention provides a costimulation blockade therapy for use in treating or preventing an autoimmune disease in a subject, which subject has been identified or determined as suitable for treatment with costimulation blockade therapy by the method according to the invention.

In a further aspect, the present invention provides a costimulation blockade therapy for use in treating or preventing an autoimmune or inflammatory disease in a subject, wherein the subject has been identified as suitable for treatment with the costimulation blockade therapy by determining the profile of B helper T cells in a sample from the subject, optionally further wherein the frequency of at least one of naïve T cells and/or regulatory T cells (Treg) is determined in the sample from the subject.

In some embodiments, the profile of B helper T cells is determined using at least one marker on CD4⁺ T cells selected from the group consisting of CXCR5, ICOS, PD-1, CD45RA, CD127, CCR7 and/or CD25.

In a further aspect, the present invention provides a computer-readable medium comprising instructions that when executed cause one or more processors to perform the method of the invention.

In a further aspect, the present invention provides an apparatus comprising:
(a) profile determination circuitry to determine the profile of B helper T cells in a sample from a subject with an autoimmune or inflammatory disease; and
(b) subject identification circuitry to identify, based on the profile determination circuitry, a suitability of the subject for treatment with costimulation blockade therapy.

In some embodiments, the frequency of at least one B helper T cell phenotype is determined, optionally wherein the at least one B helper T cell phenotype is selected from the group consisting of ICOS⁻PD-1⁻ follicular helper T cells (Tfh), ICOS⁺ Tfh, CCR7⁻PD-1⁺ Tfh, CXCR5⁺ICOS⁺ T cells, CXCR5⁻ICOS⁺ T cells, ICOS⁺PD-1^{high} Tfh, ICOS⁻PD-1⁻ memory T cells, ICOS⁻PD-1⁺ memory T cells and CXCR5⁺ naïve T cells. The frequency of at least three B helper T cell phenotypes may be determined. The at least three B helper T cell phenotypes may be ICOS⁻PD-1⁻ Tfh, ICOS⁺ Tfh and CCR7⁻PD-1⁺ Tfh.

In some embodiments, the method further comprises determining the frequency of at least one of naïve T cells and/or regulatory T cells (Treg) in the sample from the subject.

In some embodiments:
(a) a higher frequency of ICOS⁻PD-1⁻ Tfh;
(b) a lower frequency of ICOS⁺ Tfh;
(c) a lower frequency of CCR7⁻PD-1⁺ Tfh;
(d) a lower frequency of CXCR5⁺ICOS⁺ T cells;
(e) a lower frequency of CXCR5⁻ICOS⁺ T cells;
(f) a lower frequency of ICOS⁺PD-1^{high} Tfh;
(g) a higher frequency of ICOS⁻PD-1⁻ memory T cells;
(h) a higher frequency of ICOS⁻PD-1⁺ memory T cells;
(i) a lower frequency of CXCR5⁺ naïve T cells;
(j) a higher frequency of naïve T cells; and/or
(k) a higher frequency of Treg,

in comparison to a reference frequency is indicative of response to the treatment. In some embodiments, the reciprocal frequency of one or more cell phenotypes described in (a)-(k) above in comparison to a reference frequency is indicative of non-response to the treatment. The reference frequency may be from:
(a) a population of subjects who are non-responsive to the costimulation blockade therapy; and/or
(b) a population of subjects who are responsive to the costimulation blockade therapy.

In some embodiments, a frequency of one or more cell phenotypes described in (a)-(k) above in comparison to a reference frequency from a population of subjects who are non-responsive to the costimulation blockade therapy is indicative of response to the treatment.

In some embodiments, the reciprocal frequency of one or more cell phenotypes described in (a)-(k) above in comparison to a reference frequency from a population of subjects who are responsive to the costimulation blockade therapy is indicative of non-response to the treatment.

In some embodiments, at least one predictive modelling approach is used to identify the subject suitable for treatment with costimulation blockade therapy or to predict or determine whether the subject will respond to treatment with costimulation blockade therapy. The at least one predictive modelling approach may be selected from, for example, gradient boosting, random forests, support vector machines and logistic regression.

In some embodiments, populations of subjects grouped according to clinical response are used as inputs to the predictive modelling approach.

In some embodiments, the autoimmune or inflammatory disease is selected from the group consisting of type 1 diabetes, rheumatoid arthritis, psoriatic arthritis, juvenile idiopathic arthritis, Sjogren’s syndrome, Graves’s Disease, Myasthenia Gravis, inflammatory vascular diseases, glomerulonephritis and diabetic nephropathy.

In some embodiments, the autoimmune disease is type 1 diabetes.

In some embodiments, the autoimmune disease is rheumatoid arthritis.

In some embodiments, the sample is a blood sample.

In some embodiments, the costimulation blockade therapy is CD28 costimulation blockade therapy. The CD28 costimulation blockade therapy may be selected from the group consisting of a CTLA-4-Ig fusion protein, such as Abatacept, Belatacept and MEDI5265; an anti-CD28 antagonist antibody, such as lulizumab; and FR104.

In some embodiments, the subject is a human.

In some embodiments, the profile of B helper T cells is determined by flow cytometry.

In some embodiments, determining the profile of B helper T cells in the sample is carried out:
(a) prior to the onset of symptoms of the autoimmune or inflammatory disease;
(b) while the subject is showing symptoms of the autoimmune or inflammatory disease;
(c) prior to the use of costimulation blockade therapy to treat the autoimmune or inflammatory disease; and/or
(d) during and/or after the use of costimulation blockade therapy to treat the autoimmune or inflammatory disease.

### BRIEF DESCRIPTION OF THE FIGURES

FIG. 1: Abatacept decreases Tfh during an ongoing autoimmune response in mice. Abatacept or Control-Ig were injected every two to three days i.p. into 6-8 week old DO11.10 x RIP-mOVA mice. At day 11, pancreas-draining lymph nodes (panLN) and spleens were harvested for analysis. (a) Representation of treatment scheme. Collated data for frequencies (b) and absolute numbers (c) of Tfh cells in gated CD4⁺ cells. Data are compiled from two independent experiments; n=10 mice in each treatment group. Mean + SD are shown. Mann-Whitney U test; ***, p < 0.001; **, p < 0.01.

FIG. 2: Preserved C-peptide response in patients receiving Abatacept C-peptide AUC per time point and treatment as % of screening C-peptide AUC for all patients. Abatacept, n=31-34 patients; Placebo, n=14 patients. Mann-Whitney U test; **, p < 0.01; *, p < 0.05.

FIG. 3: Gating strategy. Representative gating strategy for patient samples stained for flow cytometry. PBMC samples were thawed and stained as described in the methods. Following an initial singlet gate and a live cell gate (not shown), populations were gated as presented. Names indicated are those used in downstream analysis. CM: central memory; EM: effector memory.

FIG. 4: Abatacept decreases Tfh in new onset type 1 diabetes patients. Frozen PBMC samples from recent onset T1D patients that received Abatacept or placebo were thawed and stained for flow cytometry analysis. Samples were taken at baseline, one year and two years after treatment initiation. (a) Collated data for Tfh (CD45RA⁻CXCR5⁺) frequencies in CD3⁺CD4⁺ cells from recipients of Abatacept (left) or placebo (right). (b) Principal component analysis on population frequencies obtained from flow cytometry analysis. Analysis was performed on all samples simultaneously and split into treatment groups for visualisation purposes, (c) Contributions of individual populations to PC1. (d) Collated data for ICOS⁺PD-1⁺ and CCR7⁻PD-1⁺ frequencies in CD4⁺CD45RA⁻CXCR5⁺ cells. Abatacept, n = 34 patients; Placebo, n = 13 patients (Year 1) or 14 patients (Baseline and Year 2). Wilcoxon signed-rank test; ****, p < 0.0001; ns, not significant.

FIG. 5: Minimal impact of Abatacept treatment on Tfh skewing in terms of CXCR3 and CCR6 expression. Additional frozen PBMC samples from recent onset T1D patients that received Abatacept or placebo were thawed and stained for flow cytometry analysis of Tfh skewing. (a) Collated data for Tfh (CD45RA⁻CXCR5⁺) frequencies in CD3⁺CD4⁺ cells from recipients of Abatacept (left) or placebo (right) in new cohort. (b) Collated data for ICOS⁺PD-1⁺ frequencies in Tfh cells from recipients of Abatacept or placebo in new cohort. (c) Collated data for frequencies of indicated populations of CXCR3 and CCR6 expressing Tfh cells in Abatacept and placebo treated individuals. Abatacept, n=20 patients; Placebo, n=8 patients. Wilcoxon signed-rank test; ****, p < 0.0001; ***, p < 0.001; **, p < 0.01; *, p < 0.05; ns, not significant.

FIG. 6: Data-driven analysis reveals additional Abatacept-sensitive populations in type 1 diabetes patients. CellCnn analysis followed by k-means clustering of filter-specific cells was applied to flow cytometry data of samples taken at baseline and two years after Abatacept or placebo treatment initiation. (a) Frequency of filter specific cells in each analysed sample. (b) t-SNE projection of down-sampled, pooled flow cytometry data of all samples used for CellCnn analysis. K-means clusters of filter-specific cells are highlighted. (c) Representative flow cytometry overlays of cluster-specific cells (colour) on original flow cytometry data (grey). Examples shown are from a baseline sample. (d) Frequency of cluster-specific cells in each analysed sample. (e) Collated data for frequency of manually gated T-peripheral helper cells (ICOS+PD-1+CXCR5-CD45RA- in CD4+CD3+). Abatacept, n = 34 patients; Placebo, n = 13 patients (Year 1) or 14 patients (Baseline and Year 2). Wilcoxon signed-rank test; ****, p < 0.0001; ns, not significant.

FIG. 7: Cell clusters identified by data-driven analysis correspond to known cell subsets. Cell clusters identified by CellCnn and k-means clustering to be significantly reduced in samples from Abatacept-treated individuals were overlaid onto flow cytometry data in order to infer identity. Plots show representative overlays of k-means clusters (colour) on original flow cytometry data (grey). Examples shown derive from a baseline sample.

FIG. 8: “Tph” and “ICOS+naive” cells are elevated in a mouse model of diabetes and sensitive to costimulation blockade. Cells isolated from panLN and spleens were stained with a panel of markers to identify Tph (CD4⁺CD45RB⁻CXCR5⁻ICOS⁺PD-1⁺) and ICOS⁺ naïve T cells (CD4⁺CD45RB⁺ICOS⁺). Representative flow cytometry plots for gating strategy of Tph (a) and ICOS⁺ naïve T cells (d) in spleen. Collated data for frequencies (top) and absolute numbers (bottom) of Tph (b) and ICOS⁺ naïve T cells (e) in panLN (left) and spleen (right) of DO11 and DO11 x RIP-mOVA mice. (c,f) DO11 x RIP-mOVA mice were treated with Abatacept and Control-lg according to treatment scheme depicted in FIG. 1. Shown are collated data for frequencies (top) and absolute numbers (bottom) of Tph (c) and ICOS⁺ naïve T cells (f) in panLN (left) and spleen (right). Data are compiled from 2-4 independent experiments; n=6-9 mice. Mean + SD are shown. Mann-Whitney U test; ****, p < 0.0001; ***, p < 0.001; **, p < 0.01; *, p < 0.05.

FIG. 9: Tph cells identified through CellCnn display marker expression consistent with Tph profile. Frozen PBMC samples from recent onset T1D patients that received Abatacept or placebo were thawed and analysed by flow cytometry for Tph and Tfh markers. (a) Representative gating strategy for CXCR5 vs PD-1 populations (left) and Tph previously identified through CellCnn analysis (right). (b) Collated data for frequency of cells in the CellCnn ‘Tph’ gate. (c) Expression of Tph markers on “Tph” identified by CellCnn compared with classically identified CXCR5⁻PD⁻1^{hi} Tph gated as shown in (a). Data was obtained from baseline samples. (d,e) CellCnn analysis of samples identifies a cluster of Tph-phenotype cells. Shown is expression of indicated markers within cluster (green) and all cells (grey) of representative sample (d) and frequency of cluster-specific cells in Abatacept- or Placebo-treated T1D patients (e). Abatacept, n=20 patients; Placebo, n=8 patients. Mann-Whitney U test; ****, p < 0.0001; ***, p < 0.001; **, p < 0.01; *, p < 0.05; ns, not significant.

FIG. 10: Feature selection for gradient boosting model and dynamic analysis of cell populations. (a) Representative flow cytometry plots depicting manual gating strategy for the additional populations added prior to development of a predictive model. These two populations, Tph (top) and naïve ICOS⁺ T cells (bottom), were added since they were identified by CellCnn and k-means clustering to be altered in Abatacept-treated individuals. (b) Pairwise Pearson correlation comparison of all features used in gradient boosting model. A threshold of 0.95 was used to eliminate highly correlated features. (c) Time-series plots of flow cytometry gated populations contributing to gradient boosting model. Mean and 95% confidence interval are plotted (n=10 patients in each group).

FIG. 11: Baseline Tfh phenotype is associated with clinical response to Abatacept. (a) C-peptide AUC (as % of screening C-peptide AUC) of placebo treated and top 10 (at day 728) responder and non-responder Abatacept-treated patients. Abatacept, n = 9-10 patients; Placebo, n = 14 patients. (b) A gradient boosting model was constructed using nested leave-one-out cross validation to predict clinical response following Abatacept treatment. ROC curve of the predictive model is shown. (c) Features ranked by importance for predicting clinical response following Abatacept treatment. Black lines represent 95% confidence intervals. (d) Frequencies of indicated flow cytometry gated populations at baseline (n=10 patients in each group). (a) ANOVA with Bonferroni correction; (d) Mann-Whitney U test; ****, p < 0.0001; ***, p < 0.001; **, p < 0.01; *, p < 0.05; ns, not significant.

FIG. 12: Data-driven analysis identifies cell signatures linked to clinical response to Abatacept. CellCnn analysis followed by k-means clustering of filter-specific cells was applied to flow cytometry data of samples taken at baseline from top 10 responder and non-responder Abatacept treated patients. (a) t-SNE projection of down-sampled, pooled flow cytometry data of all samples used for CellCnn analysis. Filter-specific cells for responder and non-responder filter are highlighted. (b) Frequencies of filter-specific cells in each sample for responder and non-responder filter. (c) Frequencies and representative flow cytometry overlays for clusters found in non-responder filter-specific cells. (d) Frequencies and representative flow cytometry overlays for clusters found in responder filter-specific cells. (e) Histograms of marker expression of filter-specific cells (yellow; non-responder, blue; responder) or all cells (grey). n=10 patients in each group; (b), (c) and (d) Mann-Whitney U test; (e) Kolmogorov-Smirnov (ks) test; **, p < 0.01; *, p< 0.05. All representative overlay plots are from the same baseline sample.

FIG. 13: Visualisation and frequencies of clusters identified by CellCnn that are linked to clinical response to Abatacept. Clustering results of CellCnn Responder vs Non-Responder comparison. t-SNE plot of marker expression and cluster assignment on selected cells (Responder vs Non-Responder comparison). (a, c): t-SNE projection of down-sampled, pooled flow cytometry data of all samples used for CellCnn analysis. K-means clusters or indicated marker expression of non-responder (a) and responder (c) filter-specific cells are highlighted. (b, d): Frequency of cluster-specific cells in each analysed sample for non-responder (b) and responder (d) filters. n=10 patients in each group; Mann-Whitney U test; **, p < 0.01; *, p < 0.05; ns, not significant.

FIG. 14: Cell clusters identified by data-driven analysis overlay manually gated cell populations. CellCnn and k-means clustering were used to identify populations that differed between individuals showing a good or poor response to Abatacept. Identified populations were then overlaid onto manually gated flow cytometry plots. (a) Representative overlays of cells belonging to ICOS⁺ PD-1^{hi} Tfh (left) and ICOS^{int}PD-1^{lo} Tfh (right) CellCnn clusters (red) on manual gating for ICOS⁺ Tfh cells (grey). (b) Representative overlay of ICOS⁺PD1^{hi} Tfh CellCnn cluster (red) on CCR7⁻PD-1⁺ Tfh gate (grey) (left). Collated data showing frequency of CellCnn cluster ICOS⁺PD-1^{hi} Tfh that falls within manual CCR7⁻PD1⁺ Tfh gate (right). n=20 patients. Mean ± SD are plotted in red. (c) Representative overlay of cells belonging to ICOS⁻PD-1⁻ Tfh CellCnn cluster (red) on manual gating for ICOS⁻PD-1⁻ Tfh cells (grey). Examples shown are from a baseline sample.

FIG. 15: Analysis of response to Abatacept in mouse model of autoimmune diabetes reveals similar trends to human data. Blood glucose of DO11 x RIP-mOVA mice was monitored and mice with blood glucose between 180 and 290 mg/dL were treated with Abatacept every two to three days for four weeks. Blood glucose was monitored, and Responder and Non-Responder mice were identified based on final blood glucose reading. (a) Shown are blood glucose readings of all treated mice over the treatment period. Responders and Non-Responders are highlighted in blue and yellow, respectively. Cut-offs used are highlighted in corresponding colour. (b) Baseline bleeds were stained for flow cytometry analysis and gated in a similar way to human samples, substituting CD45RB for CD45RA. The gradient boosting model used in FIG. 8 was applied to this data after removal of highly correlated features. Features ranked by importance and ROC curve of the predictive model are shown. (c,d,e) CellCnn analysis was applied to baseline samples of Responders and Non-Responders. t-SNE projection of down-sampled, pooled flow cytometry data of all samples used for CellCnn analysis (c), frequencies of filter-specific cells in each sample for Responder and Non-Responder filter (d) and histograms of marker expression of filter-specific cells (yellow; non-responder, blue; responder) or all cells (grey) (e) are shown. n=6-7 mice; (d) Mann-Whitney U test; (e) Kolmogorov-Smirnov (ks) test; *, p < 0.05.

FIG. 16: An example of an apparatus in accordance with the invention. The arrow shows the transmission of the profile of B helper T cells from the profile determination circuitry to the subject identification circuitry.

### DETAILED DESCRIPTION OF THE INVENTION

### CD4⁺ T Cells

The present invention is predicated upon the surprising finding that CD4⁺ T cell profiles could be used to derive a model that could predict clinical response to costimulation blockade. Analysis of CD4⁺ T cell profiles, and in particular B helper T cell analysis, therefore represents a new stratification tool, permitting the identification of individuals most likely to benefit from costimulation blockade.

Naïve T cells are T cells that have differentiated in the bone marrow and successfully undergone central selection in the thymus. Among these are the naïve forms of helper T cells (CD4⁺ T cells) and cytotoxic T cells (CD8⁺ T cells). A naïve T cell has not encountered its cognate antigen within the periphery, unlike activated or memory T cells. Therefore, naïve T cells can response to novel pathogens that the immune system has not yet encountered and play an essential role in the continuous response of the immune system to unfamiliar pathogens. Naive T cells are commonly characterized by the surface expression of CD62L and CCR7; the absence of the activation markers CD25, CD44 or CD69; and the absence of memory CD45RO isoform. They also express functional IL-7 receptors, consisting of subunits IL-7 receptor-α, CD127, and common-y chain, CD132.

Regulatory T cells (Tregs) are a specialized subpopulation of T cells that modulate the immune system, acting to suppress the immune response, thereby maintaining homeostasis and self-tolerance. Dysregulation in Treg cell frequency or functions may lead to the development of autoimmune disease. The most specific marker for Treg is FoxP3, which is localized intracellularly. Surface markers such as CD25^{high} (high molecular density) and CD127^{low} (low molecular density) serve as surrogate markers to detect Tregs in routine clinical practice. Treg also express CD4.

CD4⁺ T cells, also known as T helper cells, are a type of T cell that play an important role in the immune system. They help coordinate the immune response by stimulating other immune cells, such as macrophages, B cells, and CD8⁺ T cells, to fight infection by releasing T cell cytokines.

B helper T cells are CD4⁺ T cells that are able to provide help for B cell responses in in vitro assays. They are typically identified by staining for the markers CD3, CD4, CXCR5, ICOS and PD-1. They include follicular helper T cells (Tfh; CD3⁺CD4⁺CXCR5⁺ with variable expression of ICOS and PD-1) and peripheral-helper T cells (Tph; CD3⁺CD4⁺CXCR5⁻PD-1⁺ICOS⁺). Tfh support B cell responses within the germinal centers (GC) of secondary lymphoid tissues. Memory Tfh in the blood share TCR clonotypes with their lymphoid tissue counterparts and can home to GC in response to secondary immunisation.

Although type 1 diabetes (T1 D) has classically been considered to be a Type 1 T helper cell (Th1)-mediated pathology, the present inventors recently identified a signature of Tfh differentiation in this disease setting. It was found that murine T cells responding to a pancreatic self-antigen adopted a Tfh phenotype and that GC were formed in the pancreatic lymph nodes of mice developing diabetes. Likewise, in humans with T1D a higher proportion of blood-borne Tfh was observed within the memory compartment than in matched non-diabetic individuals, and similar data were obtained in two independent T1D patient cohorts. Subsequent studies showed that circulating cells with a Tfh phenotype were increased in children with multiple islet autoantibodies at risk of developing T1D. Thus, circulating Tfh-like cells have been associated with T1D in multiple patient cohorts, and increases in these cells may precede the development of overt disease.

The development of Tfh has been linked to CD28 costimulation. As described further herein, the present inventors tested whether Tfh were decreased by a costimulation blockade therapy (CTLA-4-lg/Abatacept) in a mouse model of diabetes and in individuals with new onset T1D. Unbiased bioinformatic analysis confirmed changes in CD4⁺ T cells, including B helper T cells, and revealed novel and sensitive biomarkers of costimulation blockade in T1D as a model autoimmune and/or inflammatory disease.

### Method of Patient Stratification

The present inventors have surprisingly found that the profile of CD4⁺ T cells, in particular B helper T cells, in patients having an autoimmune and/or inflammatory disease can be used to predict clinical response to costimulation blockade. Unexpectedly, the baseline (i.e. pre-treatment) profile of CD4⁺ T cells can be used to predict clinical response to costimulation blockade.

Accordingly, in one aspect, the present invention provides a method for identifying a subject with an autoimmune or inflammatory disease who is suitable for treatment with costimulation blockade therapy, the method comprising determining the profile of CD4⁺ T cells in a sample from the subject.

In a further aspect, the present invention provides a method for predicting or determining whether a subject with an autoimmune or inflammatory disease will respond to treatment with costimulation blockade therapy, the method comprising determining the profile of CD4⁺ T cells in a sample from the subject.

In a further aspect, the present invention provides a method for identifying a subject with an autoimmune or inflammatory disease who is not suitable for treatment with costimulation blockade therapy, the method comprising determining the profile of CD4⁺ T cells in a sample from the subject. This provides the advantage that the subject who is determined not to be suitable for treatment with costimulation blockade therapy using the present invention may subsequently be treated with a different therapy for the autoimmune or inflammatory disease which may be effective.

As used herein, the term “suitable for treatment” may refer to a subject who is more likely to respond to treatment with costimulation blockade therapy, or who is a candidate for treatment with costimulation blockade therapy.

As used herein, the term “not suitable for treatment” may refer to a subject who is less likely to respond to treatment with costimulation blockade therapy, or who is not a candidate for treatment with costimulation blockade therapy.

A subject suitable for treatment may be more likely to respond to said treatment than a subject who is determined not to be suitable using the present invention.

The profile of CD4⁺ T cells, such as the profile of B helper T cells, in the sample obtained from the subject may be compared to one or more reference frequencies. The one or more reference frequencies may be pre-determined. Using such reference frequencies, subjects may be stratified into categories which are indicative of the degree of response to treatment or the subjects’ percentage chance of response to treatment may be determined.

The CD4⁺ T cells may be B helper T cells.

Accordingly, in one aspect, the present invention provides a method for identifying a subject with an autoimmune or inflammatory disease who is suitable for treatment with costimulation blockade therapy, the method comprising determining the profile of B helper T cells in a sample from the subject.

In a further aspect, the present invention provides a method for predicting or determining whether a subject with an autoimmune or inflammatory disease will respond to treatment with costimulation blockade therapy, the method comprising determining the profile of B helper T cells in a sample from the subject.

In a further aspect, the present invention provides a method for identifying a subject with an autoimmune or inflammatory disease who is not suitable for treatment with costimulation blockade therapy, the method comprising determining the profile of B helper T cells in a sample from the subject.

In some preferred embodiments, the method comprises determining the pre-treatment profile of B helper T cells in a sample from the subject.

In some embodiments, the profile of B helper T cells is determined using at least one (suitably at least two, at least three, at least four, at least five, at least six or at least seven) marker on CD4⁺ T cells selected from the group consisting of CXCR5, ICOS, PD-1, CD45RA, CD127, CCR7 and/or CD25. In some embodiments, the profile of B helper T cells is determined using at least three (suitably, at least four, at least five, at least six or at least seven) markers on CD4⁺ T cells selected from the group consisting of CXCR5, ICOS, PD-1, CD45RA, CD127, CCR7 and/or CD25. In some embodiments, the at least three markers are CXCR5, ICOS and PD-1.

In some embodiments, the profile of B helper T cells is determined by determining the frequency of at least one B helper T cell phenotype. In some embodiments of the methods of the invention, the frequency of at least one B helper T cell phenotype is determined.

In some embodiments, the methods of the invention further comprise determining the frequency of at least one of naïve T cells and/or regulatory T cells (Treg) in the sample from the subject. Thus, in some embodiments, the frequency of naïve T cells and at least one B helper T cell phenotype is determined. In some embodiments, the frequency of Treg and at least one B helper T cell phenotype is determined. In some embodiments, the frequency of naïve T cells, Treg and at least one B helper T cell phenotype is determined.

There is evidence that diagnosis at a young age is associated with a more rapid loss of beta cells in T1D. Thus, in some embodiments, the methods of the invention further comprise using the age at diagnosis. In some embodiments, a younger age at diagnosis is indicative of non-response to treatment. Thus, in some embodiments, the frequency of at least one B helper T cell phenotype is determined and the age at diagnosis is used. In some embodiments, the frequency of at least one B helper T cell phenotype and the frequency of naïve T cells and/or Treg is determined, and the age at diagnosis is used.

The at least one (suitably at least two, at least three, at least four, at least five, at least six, at least seven, at least eight, at least nine) B helper T cell phenotype may be selected from the group consisting of ICOS⁻PD-1⁻ Tfh, ICOS⁺ Tfh, CCR7⁻PD-1⁺ Tfh, CXCR5⁺ICOS⁺ T cells, CXCR5⁻ICOS⁺ T cells, ICOS⁺PD-1^{high} Tfh, ICOS-PD-1- memory T cells, ICOS-PD-1⁺ memory T cells and CXCR5⁺ naïve T cells.

In some preferred embodiments, the frequency of at least three B helper T cell phenotypes is determined. In some embodiments, the at least three (suitably at least four, at least five, at least six, at least seven, at least eight, at least nine) B helper T cell phenotypes are selected from the group consisting of ICOS-PD-1⁻ Tfh, ICOS⁺ Tfh, CCR7⁻PD-1⁺ Tfh, CXCR5⁺ICOS⁺ T cells, CXCR5⁻ICOS⁺ T cells, ICOS⁺PD-1^{high} Tfh, ICOS-PD-1- memory T cells, ICOS-PD-1⁺ memory T cells and CXCR5⁺ naïve T cells. Preferably, the at least three B helper T cell phenotypes are ICOS⁻PD-1⁻ Tfh, ICOS⁺ Tfh and CCR7⁻PD-1⁺ Tfh. Thus, in some embodiments, the frequency of ICOS⁻PD-1⁻ Tfh, ICOS⁺ Tfh, CCR7⁻PD-1⁺ Tfh and at least one further B helper T cell phenotype is determined, wherein the at least one (suitably at least two, at least three, at least four, at least five, at least six) further B helper T cell phenotype is selected from the group consisting of CXCR5⁺ICOS⁺ T cells, CXCR5⁻ICOS⁺ T cells, ICOS⁺PD-1^{high} Tfh, ICOS-PD-1⁻ memory T cells, ICOS-PD-1⁺ memory T cells and CXCR5⁺ naïve T cells.

In some embodiments, the frequency of the following B helper T cell phenotypes is determined: ICOS⁻PD-1⁻ Tfh, ICOS⁺ Tfh, CCR7⁻PD-1⁺ Tfh, CXCR5⁺ICOS⁺ T cells, CXCR5-ICOS⁺ T cells, ICOS⁺PD-1^{high} Tfh, ICOS⁻PD-1⁻ memory T cells, ICOS-PD-1⁺ memory T cells and CXCR5⁺ naïve T cells.

In some embodiments, a higher frequency of ICOS⁻PD-1⁻ Tfh, a lower frequency of ICOS⁺ Tfh, a lower frequency of CCR7⁻PD-1⁺ Tfh, a lower frequency of CXCR5⁺ICOS⁺ T cells, a lower frequency of CXCR5⁻ICOS⁺ T cells, a lower frequency of ICOS⁺PD-1^{high} Tfh, a higher frequency of ICOS⁻PD-1⁻ memory T cells, a higher frequency of ICOS-PD-1⁺ memory T cells, a lower frequency of CXCR5⁺ naïve T cells, a higher frequency of naïve T cells and/or a higher frequency of Treg, in comparison to a reference frequency generated from a population of non-responders is indicative of response to the treatment.

In some embodiments, a lower frequency of ICOS⁻PD-1⁻ Tfh, a higher frequency of ICOS⁺ Tfh, a higher frequency of CCR7⁻PD-1⁺ Tfh, a higher frequency of CXCR5⁺ICOS⁺ T cells, a higher frequency of CXCR5⁻ICOS⁺ T cells, a higher frequency of ICOS⁺PD-1^{high} Tfh, a lower frequency of ICOS⁻PD-1⁻ memory T cells, a lower frequency of ICOS-PD-1⁺ memory T cells, a higher frequency of CXCR5⁺ naïve T cells, a lower frequency of naïve T cells and/or a lower frequency of Treg, in comparison to a reference frequency generated from a population of responders is indicative of non-response to the treatment.

In some preferred embodiments, a higher frequency of ICOS⁻PD-1⁻ Tfh, a lower frequency of ICOS⁺ Tfh and a lower frequency of CCR7⁻PD-1⁺ Tfh is indicative of response to the treatment.

The profile of CD4⁺ T cells, including B helper T cells, may be determined by methods known in the art, for example, the profile of the cells may be determined by flow cytometry, spectral cytometry, gene profiling or using antibodies. In some embodiments, the profile of CD4⁺T cells, including B helper T cells, is determined by flow cytometry.

In some embodiments, determining the profile of B helper T cells in the sample is carried out:
(a) prior to the onset of symptoms of the autoimmune or inflammatory disease;
(b) while the subject is showing symptoms of the autoimmune or inflammatory disease;
(c) prior to the use of costimulation blockade therapy to treat the autoimmune or inflammatory disease;
(d) during and/or after the use of costimulation blockade therapy to treat the autoimmune or inflammatory disease;
(e) after the use of costimulation blockade therapy to treat the autoimmune or inflammatory disease;
(f) during the use of costimulation blockade therapy to prevent the autoimmune or inflammatory disease; and/or
(g) after the use of costimulation blockade therapy to prevent the autoimmune or inflammatory disease.

In some embodiments, determining the profile of B helper T cells in the sample is carried out prior to the onset of symptoms of the autoimmune or inflammatory disease. In some embodiments, determining the profile of B helper T cells in the sample is carried out while the subject is showing symptoms of the autoimmune or inflammatory disease. In some embodiments, determining the profile of B helper T cells in the sample is carried out during and/or after the use of costimulation blockade therapy to treat and/or prevent the autoimmune or inflammatory disease.

In some preferred embodiments, determining the profile of B helper T cells is carried out prior to the use of costimulation blockade therapy to treat the autoimmune or inflammatory disease.

### Reference Frequency

The profile of CD4⁺ T cells, such as the profile of B helper T cells, in the sample obtained from the subject may be compared to one or more reference frequencies. The one or more reference frequencies may be pre-determined. Using such reference frequencies, subjects may be stratified into categories which are indicative of the degree of response to treatment or the subjects’ percentage chance of response to treatment may be determined.

A reference frequency may be generated from a population of healthy subjects and/or a population of subjects who have an autoimmune or inflammatory disease. Suitably, the reference frequency may be a threshold value or a range of values.

By “healthy subject”, it is meant, for example, that:
(i) the subject does not have an inflammatory and/or autoimmune disease; or
(ii) the subject has never had an inflammatory and/or autoimmune disease; or
(iii) the subject has recovered from an inflammatory and/or autoimmune disease; or
(iv) the subject suffers from no illness whatsoever.

In some embodiments, the reference frequency is generated from a population of subjects who have an autoimmune or inflammatory disease.

The population of subjects may comprise at least 10, 25, 50, 75, 100, 150, 200, 250, 500 or more subjects who have an autoimmune or inflammatory disease. The population may have any autoimmune or inflammatory disease, including an autoimmune or inflammatory disease as described herein. Alternatively, the population may all have the relevant or specific autoimmune or inflammatory disease of the subject in question. For example, the population may all have T1D.

Accordingly, the reference frequency may be obtained or derived from:
(a) a population of subjects who are non-responsive to the costimulation blockade therapy; and/or
(b) a population of subjects who are responsive to the costimulation blockade therapy.

In some embodiments, the reference frequency is from a population of subjects who are non-responsive to the costimulation blockade therapy.

In some embodiments, the reference frequency is from a population of subjects who are responsive to the costimulation blockade therapy.

In some embodiments, a reference frequency is generated from subjects who are non-responsive and subjects who are responsive to the costimulation blockade therapy. For example, the subject may be stratified by comparing the profile of CD4⁺ T cells, such as the profile of B helper T cells, in the sample obtained from the subject to a reference frequency from a population of subjects who are non-responsive to the costimulation blockade therapy and to a reference frequency from a population of subjects who are responsive to the costimulation blockade therapy. As such, the reference frequency may be a threshold value or a range of values.

It is within the capabilities of a clinician to determine if a subject with a given disease is responsive to costimulation blockade therapy based on amelioration of symptoms and/or disease-relevant markers following treatment.

Disease-relevant markers for specific autoimmune or inflammatory diseases are known in the art, including relative C-peptide retention, various glycaemic measures (HbA1c, time in range, hypoglycaemia, hyperglycaemia, glycaemic variability), level of insulin requirement, diabetes complications-associated biomarkers, Disease Activity Score (DAS), American College of Rheumatology composite (ACR) score, C-reactive protein, modified Rodnan Skin Score, swollen joint count and tender joint count. For example, for T1D disease-relevant markers include relative C-peptide retention, various glycaemic measures (HbA1c, time in range, hypoglycaemia, hyperglycaemia, glycaemic variability), level of insulin requirement and diabetes complications-associated biomarkers. By way of further example, for rheumatoid arthritis and arthritis-associated conditions (e.g. juvenile idiopathic arthritis, psoriatic arthritis, systemic lupus erythematosus (SLE) arthritis) disease-relevant markers include DAS, ACR, C-reactive protein, swollen joint count and tender joint count. By way of yet further example, modified Rodnan Skin Score is a disease-relevant marker for systemic sclerosis and scleroderma.

Clinical trials are increasingly performed in “at risk” individuals who are identified based on parameters including the presence of autoantibodies. In autoimmune diseases characterised by autoantibodies (including type 1 diabetes, rheumatoid arthritis, and SLE), autoantibodies typically appear long before development of overt disease. Thus, autoantibodies can be used as disease-relevant markers for specific autoimmune or inflammatory diseases. Suitably, autoantibodies can be used to identify subjects having the autoimmune or inflammatory disease or subjects at risk of disease development. For example, rheumatoid factor (RF) and anti-citrullinated protein antibodies (ACPA) appear before disease symptoms in rheumatoid arthritis, antibodies to pancreatic islet autoantigens appear before disease symptoms in type 1 diabetes, and antibodies to nuclear antigens appear before disease symptoms in SLE. Since B-helper T cells are elevated in these diseases and are required for autoantibodies to form, analysis of these cells will also have predictive value prior to the development of overt disease.

Categorisation of an individual’s clinical response (i.e. categorisation as a non-responder or a responder) may be performed at different time points following costimulation blockade, such as 6-month, 1-year or 2-years post treatment initiation.

Relative changes in symptoms and disease-relevant markers may be assessed by comparison with the symptoms and disease-relevant markers prior to treatment or with a negative control, and/or a positive control, such as a subject known to be responsive to treatment with costimulation blockade therapy. In some embodiments, clinical response to the costimulation blockade therapy is assessed by relative C-peptide retention at the 6-month, 1-year or 2-year time point following treatment using methods known in the art (see, for example, Beam et al. (2014) Diabetes, 63: 3120-3127). Suitably, relative C-peptide retention may be assessed as described herein.

Suitably, a subject showing no significant reduction or alleviation of one or more symptoms of the disease which is being treated following costimulation blockade therapy is considered to be a “non-responder”.

Suitably, a subject showing no significant improvement of one or more disease-relevant markers following costimulation blockade therapy is considered to be a “non-responder”.

In some embodiments, a subject having type 1 diabetes showing poor relative C-peptide retention (suitably, less than 50%, less than 45%, less than 40%, less than 35%, less than 30% relative C-peptide retention), at the 2-year time point following treatment is considered to be a “non-responder”. In some embodiments, a subject showing a C-peptide value at the 2-year time point following treatment initiation of less than 50% (suitably less than 45%, less than 40%, less than 35%, less than 30%) of the baseline value is considered to be a “non-responder”.

Suitably, a subject showing significant reduction or alleviation of one or more symptoms of the disease which is being treated following costimulation blockade therapy is considered to be a “responder”.

Suitably, a subject showing significant improvement of one or more disease-relevant markers following costimulation blockade therapy is considered to be a “responder”.

In some embodiments, a subject having type 1 diabetes showing good relative C-peptide retention (suitably, at least 80%, at least 85%, at least 90%, at least 95%, at least 100% relative C-peptide retention) at the 2-year timepoint following treatment is considered to be a “responder”. In some embodiments, a subject showing a C-peptide value at the 2-year time point following treatment initiation of at least 80% (suitably, at least 85%, at least 90%, at least 95%, at least 100%) of the baseline value is considered to be a “responder”.

A “non-responder” or “non-responsive” patient may be considered not suitable for treatment or not a candidate for treatment with costimulation blockade therapy using a method according to the invention.

A “high” or “higher” frequency of a specific cell phenotype as described herein may mean a number greater than the median frequency of this specific cell phenotype predicted or determined in the reference population of subjects, such as the minimum frequency of this specific cell phenotype predicted or determined to be in the upper quartile of the reference population. Suitably, a “high” or “higher” frequency of a specific cell phenotype as described herein may be defined as the contribution of this specific cell phenotype as a proportion of the total cells, i.e. a higher frequency ICOS⁺ Tfh means the contribution of ICOS⁺ Tfh as a proportion of the total Tfh, a higher proportion of CXCR5⁻ICOS⁺ T cells means the contribution of CXCR5⁻ICOS⁺ T cells as a proportion of the total T cells, etc..

A “low” or “lower” frequency of a specific cell phenotype as described herein may mean a number less than the median frequency of this specific cell phenotype predicted or determined in the reference population of subjects, such as the maximum frequency of this specific cell phenotype predicted or determined to be in the lower quartile of the reference population. Suitably, a “low” or “lower” frequency of a specific cell phenotype as described herein may be defined as the contribution of this specific cell phenotype as a proportion of the total cells.

A skilled person will appreciate that references to ““high”, “higher”, “low” or “lower” frequency of a specific cell phenotype may be context specific, and could carry out the appropriate analysis accordingly.

The frequency of a specific cell phenotype may be analysed by methods known in the art, e.g. as described herein. Suitably, the frequency of a specific cell phenotype may be analysed as described in the present Examples.

### Predictive Modelling Approach

A reference frequency for a specific cell phenotype may be determined using methods known in the art, e.g. as described herein. Suitably, at least one predictive modelling approach may be used to generate the reference frequency. At least one predictive modelling approach may be used to compare the frequency of at least one specific cell phenotype as described herein in the sample to the reference frequency. At least one predictive modelling approach may be used to predict the costimulation blockade therapy outcome of the subject, for example by using the frequency of at least one specific cell phenotype as described herein in the sample and the reference frequency.

In some embodiments, the reference frequency is generated using a predictive model. In some embodiments, the predictive model is trained on samples with a known clinical outcome.

In some embodiments, the reference frequency is a predictive model trained on samples with a known clinical outcome.

In some embodiments, the samples with a known clinical outcome are from a population of subjects who are responsive to the costimulation blockade therapy and/or from a population of subjects who are non-responsive to the costimulation blockade therapy. In some embodiments, the samples with a known clinical outcome are from a population of subjects who are responsive to the costimulation blockade therapy and from a population of subjects who are non-responsive to the costimulation blockade therapy.

Suitably, the model may be trained as described herein. The at least one specific cell phenotype is at least one B helper T cell phenotype, optionally further including naïve T cells and/or Treg, as described herein.

A prediction of clinical outcome based on a specific cell phenotype may be generated using methods known in the art, e.g. as described herein. Suitably, at least one predictive modelling approach may be used to generate the prediction. In some embodiments, at least one predictive modelling approach is used to generate a prediction of clinical outcome from an input of the frequency of at least one specific cell phenotype as described herein.

In some embodiments, at least one model trained on samples with a known clinical outcome is used to generate a prediction of clinical outcome from an input of the frequency of at least one specific cell phenotype as described herein.

In some embodiments, at least one predictive modelling approach may be used to predict the costimulation blockade therapy outcome of the subject, for example by using the frequency of at least one specific cell phenotype as described herein and a model trained on samples with a known costimulation blockade therapy outcome.

Suitably, the frequency of the at least one specific cell phenotype is determined from a sample from the subject as described herein. The at least one specific cell phenotype is at least one B helper T cell phenotype, optionally further including naïve T cells and/or Treg, as described herein. Suitably, the model may be trained as described herein.

Inputting the frequencies of the various CD4⁺ T cell populations into a predictive model as described herein and carrying out the appropriate analysis is within the capabilities of the person skilled in the art.

In some preferred embodiments, at least one predictive modelling approach is used to identify the subject suitable for treatment with costimulation blockade therapy.

Examples of predictive modelling approaches which may be used include gradient boosting, random forests, support vector machines and logistic regression. In some embodiments, populations of subjects grouped according to clinical response are used as inputs to the predictive modelling approach. Suitably, the inputs are a population of responders and a population of non-responders. Suitably, each population comprises at least 10 subjects. Without wishing to be bound by theory, the feedback provided by the known population(s) provides the advantage that it trains the model to work on future populations.

In one particular example in which gradient boosting is used, a population of subjects with the best clinical response (responders) and a population of subjects with the poorest clinical response (non-responders) are used to build the predictive model. Suitably, each population comprises at least 10 subjects. Pairwise correlation comparisons are conducted between features to identify and remove features that are highly correlated (Pearson correlation coefficient greater than 0.95), ensuring feature importance could be legitimately interpreted from the gradient boosting model: where two features are shown to be highly correlated, the one least correlated with outcome is removed from the set of features used to build the predictive model. The gradient boosting model is constructed using nested leave-one-out cross validation: each of the n patients is iteratively removed from the dataset and kept aside for testing purposes, the remaining n-1 baseline samples are used for model training and hyperparameter (learning rate, maximum depth and number of estimators) tuning using 3-fold cross validation, the optimal model from this training process is then used to make a prediction on the “left-out” sample, and feature weights are recorded. Alternative cross validation strategies are known in the art. Selecting a suitable cross validation strategy for use in the methods described herein is within the ambit of the person skilled in the art. The determination of suitable features for use in the predictive model is within the capabilities of the person skilled in the art. Suitably, the features are as described herein.

### Computer-Readable Medium and Apparatus

In a further aspect, the present invention provides a computer-readable medium comprising instructions that when executed cause one or more processors to perform the method of patient stratification as described herein.

In a further aspect, the present invention provides a non-transitory computer-readable medium comprising instructions that when executed cause one or more processors to perform the method of patient stratification as described herein.

A computer readable medium may include non-transitory media such as physical storage media including storage discs and solid state devices. A computer readable medium may also or alternatively include transient media such as carrier signals and transmission media. An example computer-readable storage medium is a non-transitory memory device. A memory device includes memory space within a single physical storage device or memory space spread across multiple physical storage devices.

In a further aspect, the present invention provides an apparatus (10) comprising:
(a) profile determination circuitry (11) to determine the profile of B helper T cells in a sample from a subject with an autoimmune or inflammatory disease; and
(b) subject identification circuitry (12) to identify, based on the profile determination circuitry, a suitability of the subject for treatment with costimulation blockade therapy.

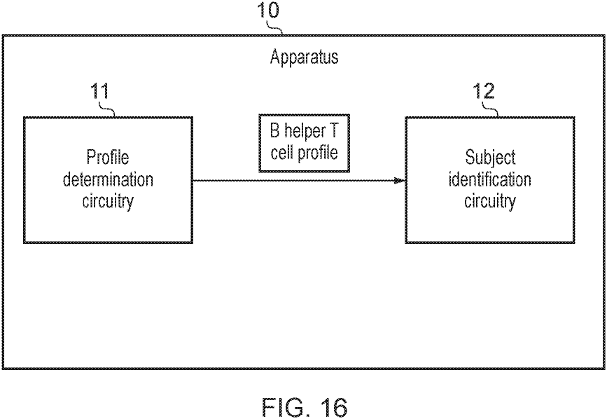

The profile determination circuitry (11) and subject identification circuitry (12) may be dedicated circuitry elements configured to perform the described functionality. Alternatively or additionally, at least one circuitry element may be implemented with semi-dedicated circuitry units such as field-programmable gate arrays and/or application-specific integrated circuits. Alternatively or additionally, at least one such circuitry element may be implemented as a conceptual or logical function of a general-purpose processing circuit such as a central processing unit or graphics processing unit. FIG. 16 shows an example apparatus in accordance with the invention.

### Autoimmune and/or Inflammatory Disease

The present invention provides a method for identifying a subject with an autoimmune and/or inflammatory disease who is suitable for treatment with costimulation blockade therapy.

The present invention further provides a method for predicting or determining whether a subject with an autoimmune and/or inflammatory disease will respond to treatment with costimulation blockade therapy.

The present invention yet further provides methods of treating or preventing an autoimmune and/or inflammatory disease in a subject.

A method for the prevention of an autoimmune and/or inflammatory disease relates to the prophylactic use of the costimulation blockade therapy. Herein the costimulation blockade therapy may be administered to a subject who has not yet contracted or developed an autoimmune and/or inflammatory disease and/or who is not showing any symptoms of the disease to prevent or impair the cause of the disease or to reduce or prevent development of at least one symptom associated with the disease.

A method for the treatment of an autoimmune and/or inflammatory disease relates to the therapeutic use of the costimulation blockade therapy. Herein the costimulation blockade therapy may be administered to a subject having an existing disease or condition in order to lessen, reduce or improve at least one symptom associated with the disease and/or to slow down, reduce or block the progression of the disease.

The subject may have a predisposition for, or be thought to be at risk of developing, an autoimmune or inflammatory disease.

The methods of the invention may be used to treat and/or prevent a disease such as an inflammatory disease or an autoimmune disease.

The disease may involve or be associated with Tfh differentiation and/or increases in circulating cells having a Tfh phenotype. Suitably, the circulating cells may have a Tfh phenotype as described herein. The disease may involve or be associated with CD28 costimulation.

The disease may be suitable for treatment with costimulation blockade, such as CD28 costimulation blockade. The natural regulator of CD28 is the inhibitory receptor CTLA-4, and a soluble version of this molecule has been developed for therapeutic use. Soluble CTLA-4 (a fusion protein with human immunoglobulin; CTLA-4-lg) is widely used in autoimmune and/or inflammatory diseases. T cells, including autoreactive T cells, are key players in autoimmune and inflammatory diseases. Thus, autoimmune and inflammatory diseases are suitable for treatment with costimulation blockade, such as CD28 costimulation blockade.

The disease may, for example, be one of the following: type 1 diabetes, rheumatoid arthritis, psoriatic arthritis, juvenile idiopathic arthritis, juvenile dermatomyositis, Sjogren’s syndrome, Graves’s Disease, Myasthenia Gravis, glomerulonephritis, diabetic nephropathy, primary biliary cirrhosis, autoimmune hepatitis, vitiligo, alopecia areata, multiple sclerosis, systemic lupus erythematosus (SLE) including SLE arthritis, psoriasis, scleroderma, systemic sclerosis including cutaneous systemic sclerosis, IgG4-related disease, uveitis, graft versus host disease, CTLA-4 haplosufficiency or diseases associated with CTLA-4-pathway dysfunction (e.g. individuals with LRBA mutations), myositis and myositis-related interstitial lung disease and inflammatory vascular diseases, such as atherosclerosis, autoimmune vasculitis, giant cell arteritis, granulomatosis with polyangiitis, Wegener’s Granulomatosis, ANCA-associated vasculitis.

In some embodiments, the disease is selected from the group consisting of type 1 diabetes, rheumatoid arthritis, psoriatic arthritis, juvenile idiopathic arthritis, juvenile dermatomyositis, Sjogren’s syndrome, Graves’s Disease, Myasthenia Gravis, glomerulonephritis, diabetic nephropathy and SLE including SLE arthritis.

In some embodiments, the disease is type 1 diabetes.

The disease may be a disease characterised by a period during which an individual is “at risk” of developing overt disease. For example, type I diabetes is characterised by a pre-diabetic period (corresponding to Stage 1 and Stage 2 diabetes) during which an individual is at risk of developing overt disease, known as Stage 3 diabetes. The period during which an individual is “at risk” of developing overt disease may be determined using disease-relevant markers which are present prior to the onset of overt disease. For example, autoantibodies can be used to assess risk of disease development. Suitably, the disease is selected from the group consisting of type 1 diabetes, SLE, rheumatoid arthritis, juvenile idiopathic arthritis and other rheumatic diseases.

The disease may be an autoimmune disease characterised by autoantibodies. Suitably, the disease is selected from the group consisting of type 1 diabetes, SLE, rheumatoid arthritis, juvenile idiopathic arthritis and other rheumatic diseases.

In some embodiments, the disease is rheumatoid arthritis.

### Subject

The subject may be a mammalian subject such as a human. The subject may be any age, gender or ethnicity.

The subject may have an inflammatory and/or autoimmune disease, or be thought to be at risk from contracting or developing an inflammatory and/or autoimmune disease, because of, for example, family history of the disease or the presence of genetic or phenotypic (e.g. biomarkers) associated with the disease.

Clinical trials are increasingly performed in “at risk” individuals who are identified based on parameters including the presence of autoantibodies. In autoimmune diseases characterised by autoantibodies (including type 1 diabetes, rheumatoid arthritis, and SLE), autoantibodies typically appear long before development of overt disease. Thus, autoantibodies can be used to identify subjects having a disease and/or to assess risk of disease development. For example, rheumatoid factor (RF) and anti-citrullinated protein antibodies (ACPA) appear before disease symptoms in rheumatoid arthritis, antibodies to pancreatic islet autoantigens appear before disease symptoms in type 1 diabetes, and antibodies to nuclear antigens appear before disease symptoms in SLE. Since B-helper T cells are elevated in these diseases and are required for autoantibodies to form, analysis of these cells will also have predictive value prior to the development of overt disease.

Type 1 diabetes has recently been redefined, incorporating the concept that the disease process begins long before the clinical diagnosis of diabetes. According to the new definitions, having multiple islet autoantibodies and normal glucose tolerance is classed as Stage 1, having multiple autoantibodies and abnormal glucose tolerance is classed as Stage 2, and having clinical symptoms of type 1 diagnosis is classed as Stage 3. Pre-diabetics may be defined as individuals that do not yet have overt diabetes. Thus, Stage 1 and Stage 2 individuals are pre-diabetic whilst Stage 3 individuals have developed overt disease.

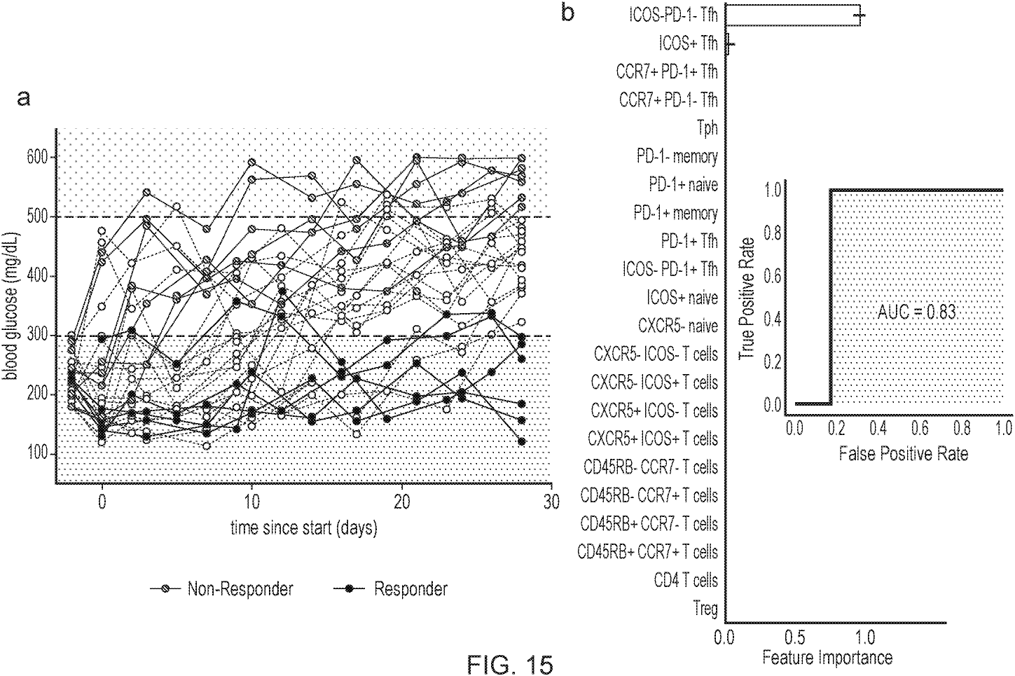

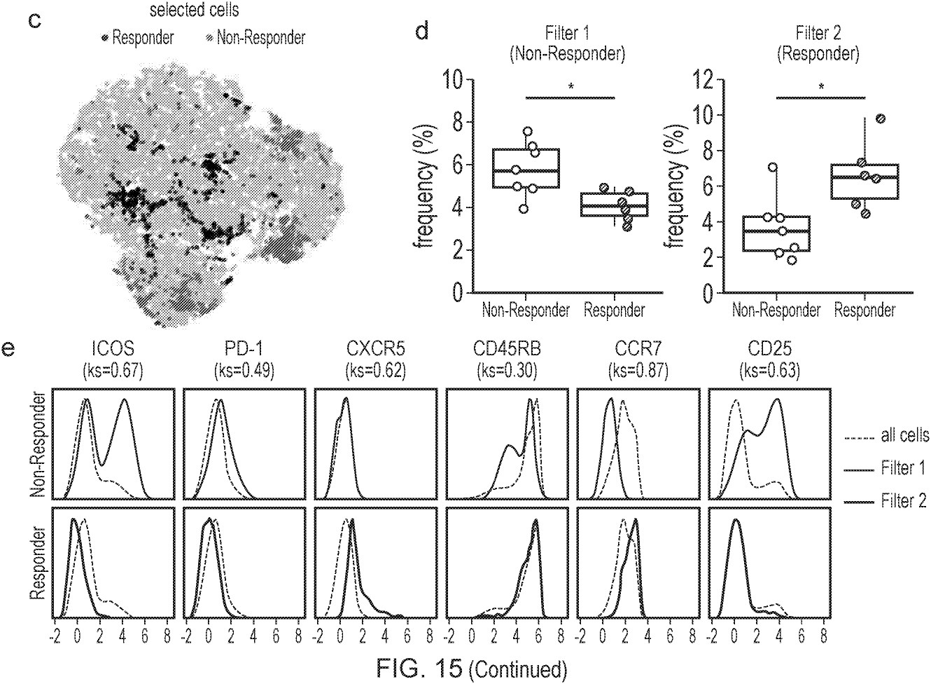

In the mouse model of autoimmune diabetes used herein, because disease develops over weeks rather than years, it is not possible to distinguish between Stage 1 and Stage 2 diabetes. However, mice with abnormal glucose homeostasis that do not yet have overt diabetes (pre-diabetic mice) can be identified. The inventors have demonstrated that clinical response to costimulation blockade using the methods described herein can be performed in these pre-diabetic animals, thereby providing evidence that the methods described herein can be informative prior to the development of overt disease (see FIG. 15).

The subject may be thought to be at risk from contracting or developing an inflammatory and/or autoimmune disease, because of, for example, family history of the disease or the presence of genetic or phenotypic (e.g. biomarkers) associated with the disease (e.g. autoantibodies), optionally wherein the disease is selected from the group consisting of: type 1 diabetes, rheumatoid arthritis, psoriatic arthritis, juvenile idiopathic arthritis, juvenile dermatomyositis, Sjogren’s syndrome, Graves’s Disease, Myasthenia Gravis, glomerulonephritis, diabetic nephropathy, primary biliary cirrhosis, autoimmune hepatitis, vitiligo, alopecia areata, multiple sclerosis, systemic lupus erythematosus (SLE) including SLE arthritis, psoriasis, scleroderma, systemic sclerosis including cutaneous systemic sclerosis, IgG4-related disease, uveitis, graft versus host disease, CTLA-4 haplosufficiency or diseases associated with CTLA-4-pathway dysfunction (e.g. individuals with LRBA mutations), myositis and myositis-related interstitial lung disease and inflammatory vascular diseases, such as atherosclerosis, autoimmune vasculitis, giant cell arteritis, granulomatosis with polyangiitis, Wegener’s Granulomatosis, ANCA-associated vasculitis.

In some embodiments, the disease is selected from the group consisting of type 1 diabetes, rheumatoid arthritis, psoriatic arthritis, juvenile idiopathic arthritis, juvenile dermatomyositis, Sjogren’s syndrome, Graves’s Disease, Myasthenia Gravis, glomerulonephritis, and diabetic nephropathy and SLE including SLE arthritis.

In some embodiments, the disease is type 1 diabetes.

In some embodiments, the disease is rheumatoid arthritis.

The subject may show one or more signs or symptoms of an inflammatory and/or autoimmune disease. The subject may have been previously characterised as having an inflammatory and/or autoimmune disease by other diagnostic methods.

The subject may have been determined to be a “responder” by the method of patient stratification of the present invention described herein.

The subject may have been previously treated with costimulation blockade therapy.

The methods and uses described herein may be performed to determine whether the subject is suitable for treatment with costimulation blockade therapy:
(a) prior to the onset of symptoms of the autoimmune or inflammatory disease;
(b) while the subject is showing symptoms of the autoimmune or inflammatory disease;
(c) during and/or after the use of costimulation blockade therapy to treat the autoimmune or inflammatory disease;
(d) after the use of costimulation blockade therapy to treat the autoimmune or inflammatory disease;
(e) during the use of costimulation blockade therapy to prevent the autoimmune or inflammatory disease; and/or
(f) after the use of costimulation blockade therapy to prevent the autoimmune or inflammatory disease.

In some embodiments of the methods and uses of the invention, determining whether the subject is suitable for treatment with costimulation blockade therapy is performed:
(a) prior to the onset of symptoms of the autoimmune or inflammatory disease;
(b) during the use of costimulation blockade therapy to prevent the autoimmune or inflammatory disease; and/or
(c) after the use of costimulation blockade therapy to prevent the autoimmune or inflammatory disease.

In some preferred embodiments of the methods and uses of the invention, determining whether the subject is suitable for treatment with costimulation blockade therapy is performed prior to the onset of symptoms of the autoimmune or inflammatory disease.

### Sample

Isolation of samples from a subject is common practice in the art and may be performed according to any suitable method, and such methods will be known to one skilled in the art.

The sample may be or may be derived from a biological sample, such as a blood sample, a biopsy specimen, a tissue extract or any other tissue or cell preparation from a subject.

In theory, the profile of B helper T cells can be determined according to the present invention by extracting blood cells, specifically T cells, from any tissue of the body.

The sample may be or may be derived from an ex vivo sample.

The sample may be a blood sample.

Preferably, the sample is, or is derived from blood, in particular peripheral blood.

Preferably, the sample is, or is derived from, whole blood or a fraction of whole blood.

Suitably, the sample will have been isolated from the subject prior the methods of the present invention. In other words, suitably the step of isolating the sample from the subject does not form part of the present methods.

### Costimulation Blockade Therapy

T cells are activated through multiple cell signalling pathways. These pathways include a primary recognition signal, involving interaction of their T cell receptor (TCR) with peptide-MHC complex, and additional costimulatory signals. Signalling through accessory molecules or costimulatory molecules is a critical way for the immune system to fine tune T cell activation. Thus, efficient T cell responses occur with concomitant T cell receptor antigen specific signal activation and non-antigen specific costimulatory signal activation. T-cell costimulation blockade attempts to decrease the T-cell response by inhibiting one component of T-cell activation, a costimulatory molecule, thus leading to tolerance. This inhibition is of major interest in transplant recipients and autoimmune or inflammatory diseases.

Accordingly, “costimulation blockade therapy” describes treatments which are directed at decreasing the T-cell response by inhibiting the costimulatory signal.

As used herein, “costimulation blockade therapy” may refer to any therapy which interacts with or modulates a costimulatory signalling interaction or costimulatory signalling cascade (either at an extracellular or intracellular level) in order to decrease/reduce immune cell activity (in particular T cell activity). For example the costimulation blockade therapy may prevent, reduce, minimise or inhibit T cell activation and T cell activity. The costimulation blockade therapy may decrease T cell activation by inhibiting costimulatory signalling. By “inhibit” is meant any means to prevent T cell activation by, for example, blocking at least one costimulatory signalling pathway. This can be achieved by antibodies or molecules that block receptor ligand interaction, inhibitors of intracellular signalling pathways, and compounds preventing the expression of costimulatory molecules on the T cell surface.

Suitably, the “costimulation blockade therapy” may be a therapy which interacts with or modulates a costimulatory molecule. For example, the “costimulation blockade therapy” may inhibit receptor ligand binding.

Costimulation blockade therapies are known in the art.

The costimulation blockade therapy may selected from one or more of the following: an antibody, an Ig fusion protein, a polypeptide, a peptide, a polynucleotide, a small molecule, a non-antibody scaffold, an aptamer, or combinations thereof.

The term “antibody” includes intact antibodies, fragments of antibodies, e.g., Fab, F(ab′) 2 fragments, and intact antibodies and fragments that have been mutated either in their constant and/or variable region (e.g. mutations to produce chimeric, partially humanized, or fully humanized antibodies, as well as to produce antibodies with a desired trait, e.g., enhanced CD28 binding).

The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Binding fragments include Fab, Fab′, F(ab′) 2, Fabc, Fd, dAb, Fv, single chains, single-chain antibodies, e.g. scFv, single domain antibodies, and an isolated complementarity determining region (CDR).

Antibody-like molecules include the use of CDRs separately or in combination in synthetic molecules such as SMIPs and small antibody mimetics. Specificity determining regions (SDRs) are residues within CDRs that directly interact with antigen. The SDRs correspond to hypervariable residues. CDRs can also be utilized in small antibody mimetics, which comprise two COR regions and a framework region.

One of the best-characterized costimulatory pathways includes the Ig superfamily members CD28 and CTLA-4 and their ligands CD80 and CD86. CD28 has been implicated in the provision of T cell help for antibody responses and the development of Tfh. Recently, the present inventors reported that Tfh differentiation was sensitive to the strength of CD28 engagement, and that this could be modulated by CTLA-4 (Wang, C.J., et al. (2015) Proc Natl Acad Sci USA, 112: 524-529). CD28 costimulation licences T cells for effective activation and is a key therapeutic target in autoimmunity. The natural regulator of CD28 is the inhibitory receptor CTLA-4, and a soluble version of this molecule has been developed for therapeutic use.

Soluble CTLA-4 (a fusion protein with human immunoglobulin; CTLA-4-lg) is widely used in autoimmune diseases including rheumatoid arthritis (RA), psoriatic arthritis and juvenile idiopathic arthritis. Clinical trials have also been undertaken in patients with Sjorgren’s syndrome and multiple sclerosis. In particular, CTLA-4-lgs Abatacept and Belatacept are clinically approved agents for the treatment of autoimmune diseases and renal transplantation, respectively. Abatacept is licensed for the treatment of RA, psoriatic arthritis and juvenile idiopathic arthritis.

Studies in the NOD mouse model of T1D suggested a protective effect of CTLA-4-lg in this disease setting leading to a trial of Abatacept (Orenica; Bristol-Myers Squibb) in individuals with new onset T1D. A randomised double-blind placebo controlled trial demonstrated some efficacy of Abatacept at 2 years compared with placebo, and the beneficial effects were largely maintained a year following therapy cessation, although it was clear that some individuals benefited more than others.

In some embodiments of the invention, the costimulation blockade therapy is CD28 costimulation blockade therapy. CD28 costimulation blockade therapies are known in the art and include, by way of example, a CTLA-4-lg fusion protein, such as Abatacept, Belatacept and MEDl5265; an anti-CD28 antagonist antibody, such as lulizumab; and FR104. In some preferred embodiments, the CD28 costimulation blockade therapy is a CTLA-4-lg fusion protein, such as Abatacept, Belatacept and MEDl5265. Preferably, the CD28 costimulation blockade therapy is Abatacept.

### Treatment or Prevention of an Autoimmune And/or Inflammatory Disease

The methods according to the invention as described herein may further comprise the step of administering a costimulation blockade therapy to a subject who has been identified as suitable for treatment with a costimulation blockade therapy.

Accordingly, in a further aspect, the present invention provides a method of treating or preventing an autoimmune or inflammatory disease in a subject which comprises treating a subject with or at risk of developing an autoimmune or inflammatory disease with costimulation blockade therapy, wherein the subject has been identified as suitable for treatment with the costimulation blockade therapy by determining the profile of CD4⁺ T cells in a sample from the subject as described herein.

In a further aspect, the present invention provides a method of treating or preventing an autoimmune or inflammatory disease in a subject which comprises treating a subject with or at risk of developing an autoimmune or inflammatory disease with costimulation blockade therapy, wherein the subject has been identified as suitable for treatment with the costimulation blockade therapy by determining the profile of B helper T cells in a sample from the subject as described herein.

In a further aspect, the present invention provides a method of treating or preventing an autoimmune or inflammatory disease in a subject, wherein the method comprises the following steps:
(a) identifying or determining a subject with or at risk of developing an autoimmune disease who is suitable for treatment with costimulation blockade therapy as described herein; and
(b) treating the subject with costimulation blockade therapy.

In a further aspect, the present invention provides a method of treating or preventing an autoimmune or inflammatory disease in a subject which comprises treating a subject with or at risk of developing an autoimmune or inflammatory disease with costimulation blockade therapy, which subject has been identified or determined as suitable for treatment with costimulation blockade therapy as described herein.

A subject who is suitable for treatment with costimulation blockade therapy may be identified or determined using a method of the present invention.

In some embodiments, the method further comprises using the age at diagnosis as described herein. In some embodiments, the method further comprises determining the frequency of at least one of naïve T cells and/or regulatory T cells (Treg) in the sample from the subject as described herein.

As defined herein “treatment” refers to reducing, alleviating or eliminating one or more symptoms of the disease, disorder or condition which is being treated, relative to the symptoms prior to treatment.

“Prevention” (or prophylaxis) refers to delaying or preventing the onset of the symptoms of the disease, disorder or condition. Prevention may be absolute (such that no disease occurs) or may be effective only in some individuals or for a limited amount of time.

Treatment according to the invention may also encompass the use of a costimulation blockade therapy in a subject who has been identified as suitable for treatment as described herein.

Accordingly, in a further aspect, the present invention provides a costimulation blockade therapy for use in a method of treatment or prevention of an autoimmune or inflammatory disease in a subject, the method comprising:
(a) identifying or determining a subject with or at risk of developing an autoimmune or inflammatory disease who is suitable for treatment with costimulation blockade therapy as described herein; and
(b) treating the subject with costimulation blockade therapy.

In a further aspect, the present invention provides a costimulation blockade therapy for use in treating or preventing an autoimmune disease in a subject, which subject has been identified or determined as suitable for treatment with costimulation blockade therapy as described herein.

In a further aspect, the present invention provides a costimulation blockade therapy for use in treating or preventing an autoimmune or inflammatory disease in a subject, wherein the subject has been identified as suitable for treatment with the costimulation blockade therapy by determining the profile of B helper T cells in a sample from the subject as described herein, optionally further wherein the age at diagnosis is used as described herein and/or the frequency of at least one of naïve T cells and/or Treg is determined in the sample from the subject as described herein. In some embodiments, the age at diagnosis is used as described herein and the frequency of at least one of naïve T cells and/or Treg is determined in the sample from the subject as described herein. In some embodiments, the frequency of naïve T cells and Treg is determined in the sample from the subject as described herein In some embodiments, the frequency of naïve T cells is determined in the sample from the subject as described herein. In some embodiments, the frequency of Treg is determined in the sample from the subject as described herein.

The methods and uses for treating or preventing an autoimmune or inflammatory disease according to the present invention may be performed in combination with additional therapies. In particular, the costimulatory blockade therapies according to the present invention may be administered in combination with other immunotherapies, including immunosuppressive therapies (e.g. methotrexate, prednisone, rituximab), metabolic therapies (e.g. therapies to improve beta cell function such as GLP-1R agonists), regulatory T cell therapy and antigen-specific immunotherapy. Suitably, the costimulatory blockade therapies according to the present invention may be administered in combination with methotrexate, prednisone and/or rituximab.

### Administration

In the methods of treatment or prevention of an autoimmune or inflammatory disease as described herein, the costimulation blockade therapy is administered to the subject. In some embodiments of the methods described herein, the costimulation blockade therapy is administered simultaneously, separately or sequentially with an additional therapy as described herein.

In some embodiments, the costimulation blockade therapy is administered to the subject, followed by the additional therapy. Alternatively, the two therapeutic agents may be administered simultaneously, for at least part of the treatment. For example, the subject may be given the first therapy, either as a single treatment or a course of treatment; followed by the second therapy, optionally in combination with the first therapy, either as a single treatment or a course of treatment.

Typically, a physician will determine the actual dosage which will be most suitable for an individual subject and it will vary with the age, weight and response of the particular subject. Each therapeutic agent may be administered with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s}, coating agent(s), solubilising agent(s), and other carrier agents.

Suitable the subject may be a mammal, preferably a human.

Where appropriate, the agent(s) or composition(s) can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Materials and Methods

### Patients

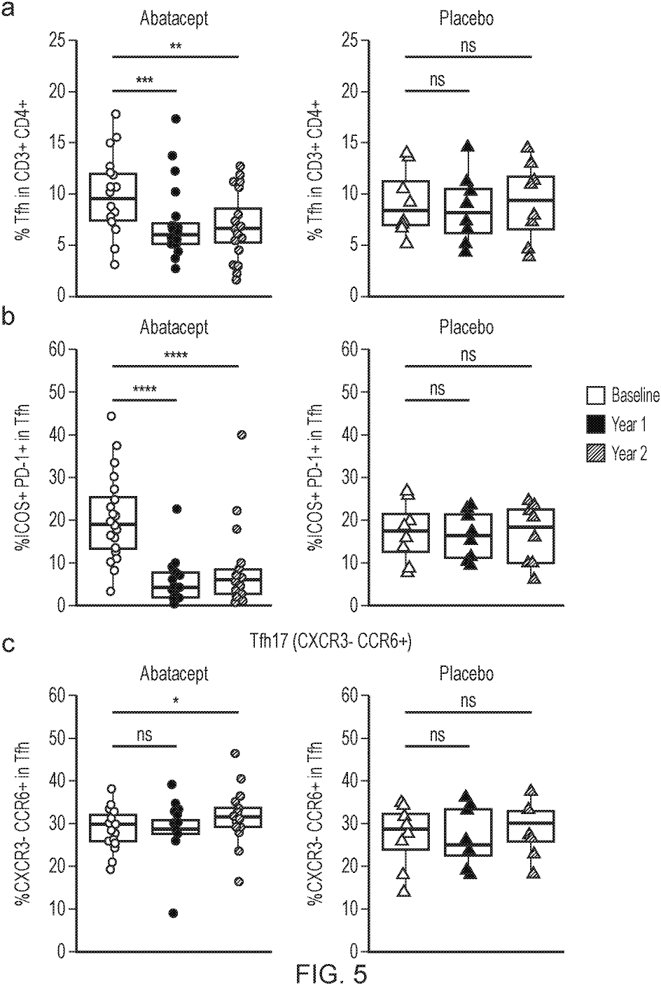

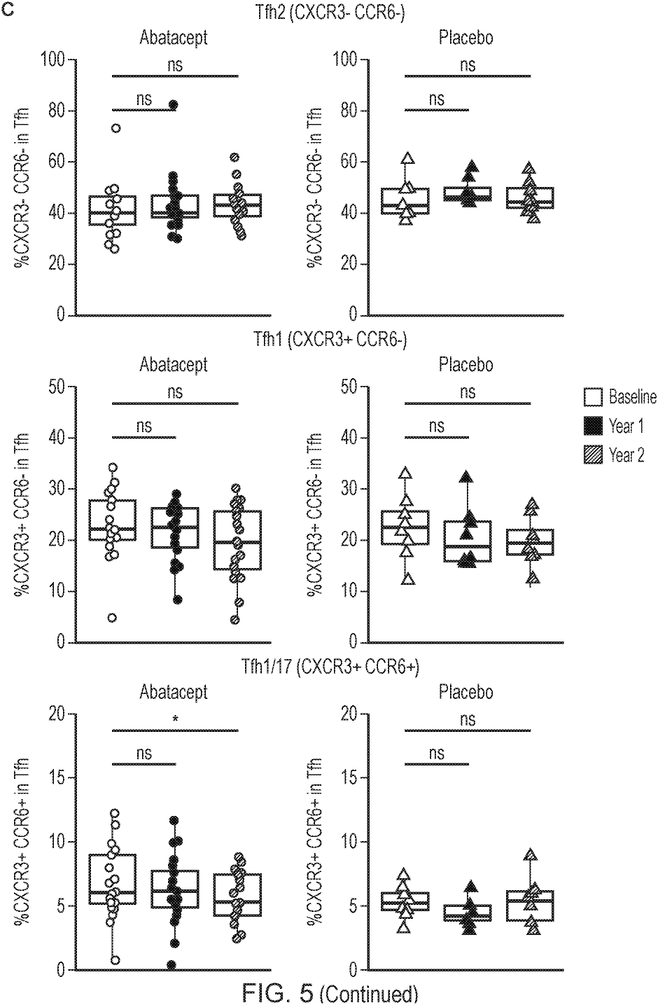

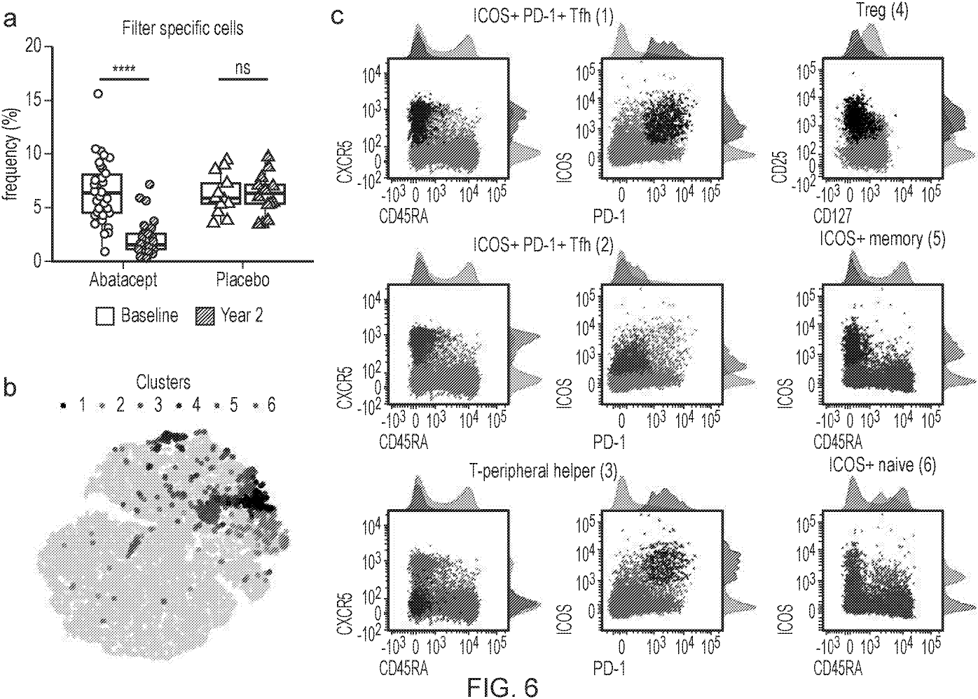

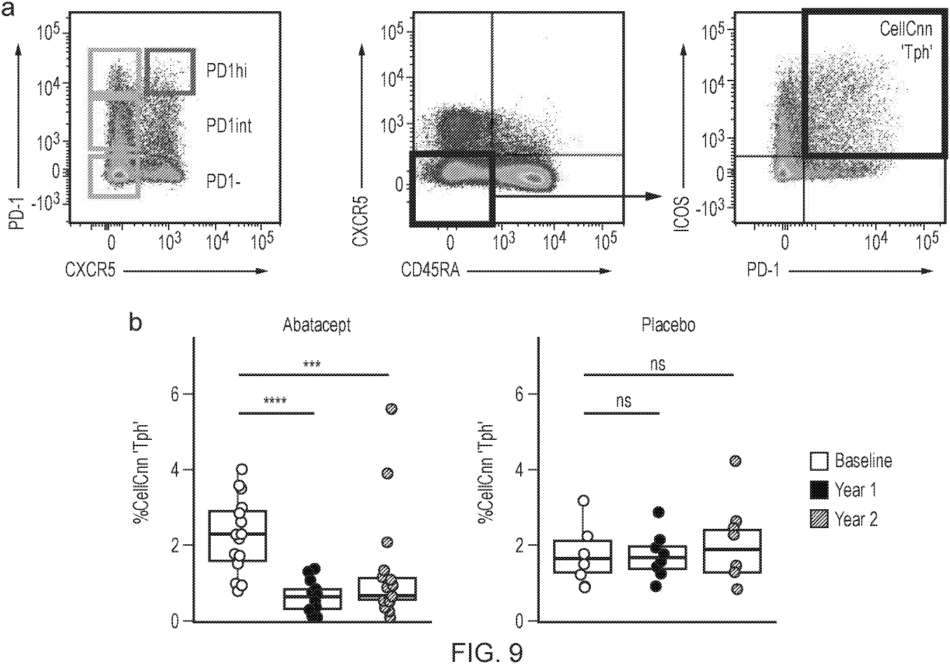

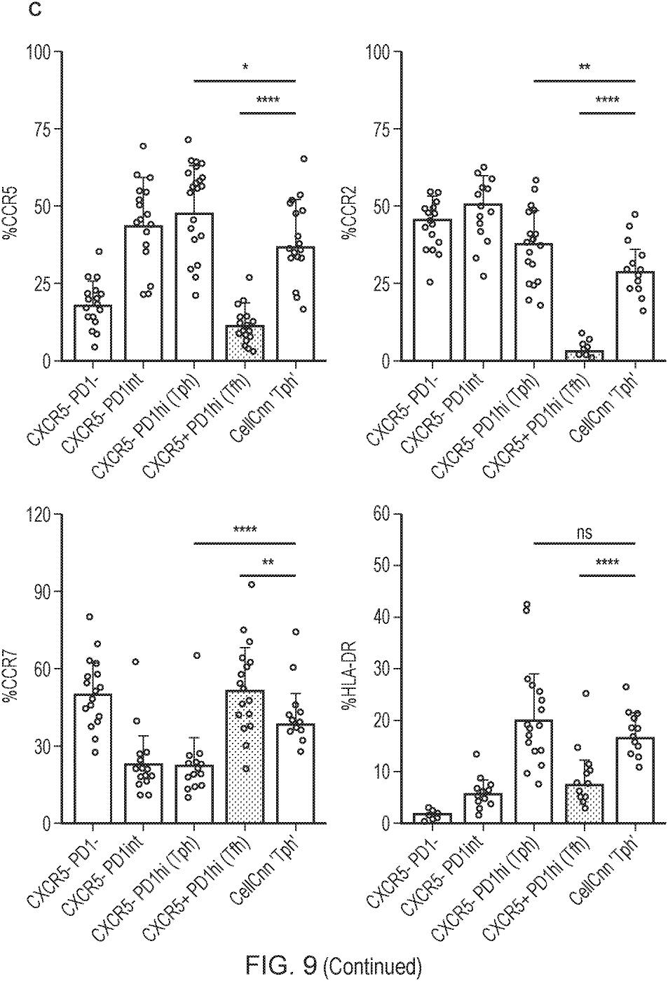

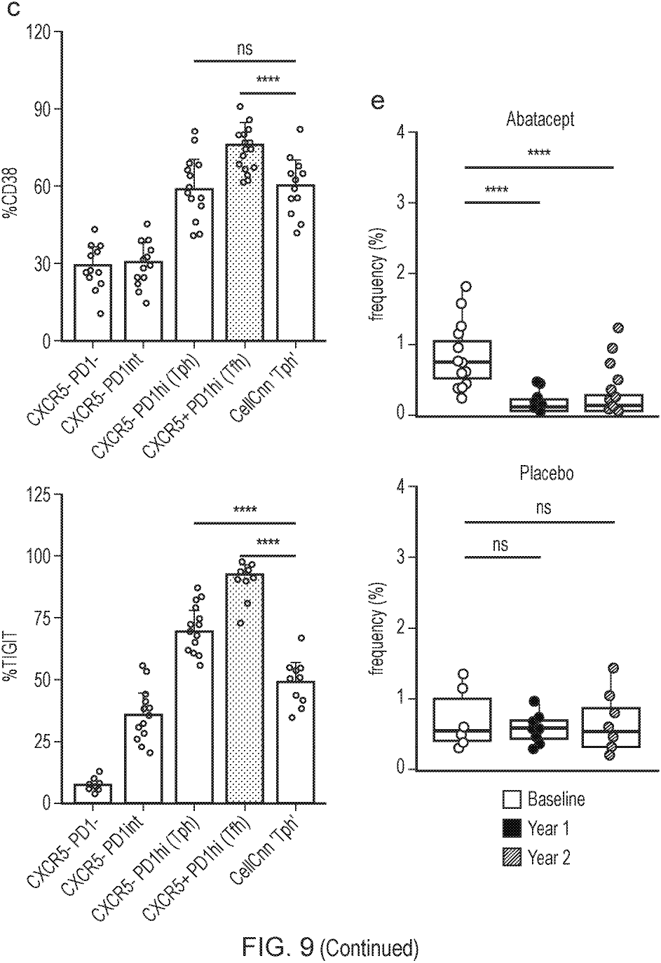

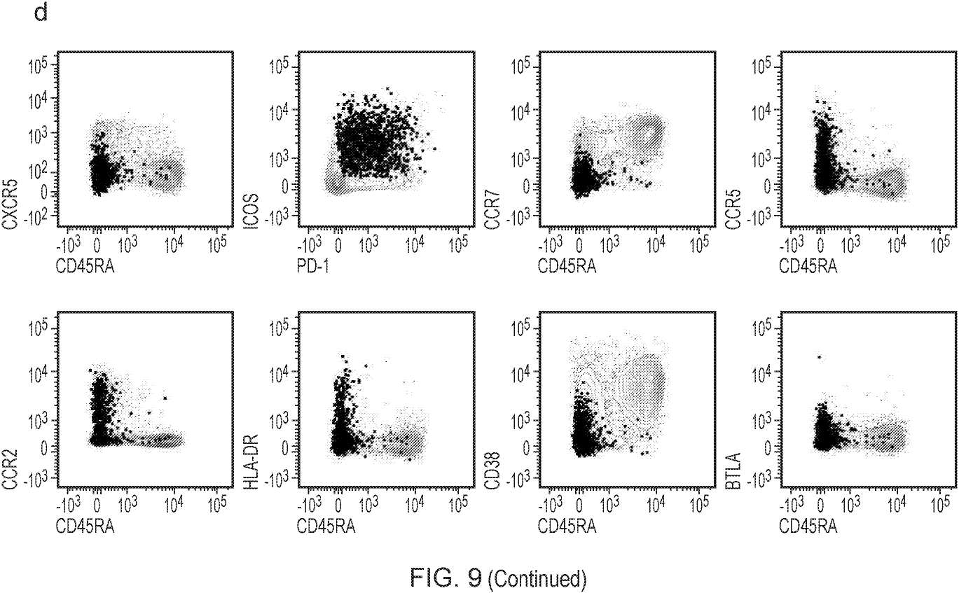

Cryopreserved PBMC samples from a clinical trial (NCT00505375) that has previously been Published (Orban, T., et al. (2011) Lancet 378: 412-419) were provided by Type 1 Diabetes TrialNet as part of the “Effects of CTLA-4 IG(Abatacept) on the Progression of Type 1 Diabetes in New Onset Subjects (TN-09)” study. Briefly, in this study individuals with recent onset T1D (diagnosed within the past 100 days) were randomised to receive CTLA4-lg (Abatacept) (10 mg/kg) or placebo (saline) intravenously on days 1, 14, 28 and subsequently once monthly for 2 years. Samples were provided from study participants at the time of screening and 12 and 24 months following treatment initiation. Data from 36 Abatacept-treated and 14 placebo-treated patients were acquired. Samples from 2 Abatacept-treated individuals were excluded from the analysis due to low data quality. For one placebo-treated patient, no 12-month sample was acquired. Samples were supplied in a blinded and randomised way in two batches separated by a break of 9 months. A further set of samples from 20 Abatacept-treated and 8 placebo-treated patients were obtained and analysed (FIG. 9, FIG. 5). Demographic and clinical data were only provided following submission of raw data files to TrialNet. To assess stimulated C-peptide secretion, four-hour mixed meal tolerance tests (MMTTs) were performed at screening and at 24 months. Additional two-hour MMTTs were conducted at 3, 6, 12 and 18 months, although for some patients C-peptide data was not available for all timepoints. For comparison across all timepoints only the first 2 hours of the 4-hour MMTTs were used.

### Mice

BALB/c DO11.10 TCR transgenic mice were obtained from The Jackson Laboratory and BALB/c CD28-/- mice from Taconic Laboratories. BALB/c RIP-mOVA mice (expressing the ovalbumin transgene under control of the rat insulin promoter, from line 296-1B) were a gift from W. Heath (The Walter and Eliza Hall Institute, Parkville, Melbourne, Australia). DO11.10 mice were crossed with RIP-mOVA mice to generate DO11 x RIP-mOVA mice. Mice were housed in individually vented cages with environmental enrichment (e.g. cardboard tunnels, paper houses, chewing blocks) at University College London Biological Services Unit. Experiments were performed in accordance with the relevant Home Office project and personal licenses following approval from the University College London Animal Welfare Ethical Review Body.

### In Vivo Experiments

For experiments using DO11x RIP-mOVA mice, 6-13 week old animals were injected i.p. with 500 µg Abatacept or control antibody. Mice were subsequently treated with 250 µg Abatacept or control antibody every 2-3 days over a period of 11 days. In this mouse model of autoimmune diabetes, because disease develops over weeks rather than years, it is not possible to distinguish between Stage 1 and Stage 2 diabetes. However, mice with abnormal glucose homeostasis that do not yet have overt diabetes (pre-diabetic) can be identified. For example, for experiments in FIG. 15, DO11xRIP-mOVA mice with a blood glucose reading between 180 and 290 mg/dL were injected i.p. with Abatacept, 500 µg for the initial dose then 250 µg three times weekly, for four weeks and blood glucose was monitored. Mouse spleen and lymph nodes were mashed to create single cell suspensions and 2-10 × 10⁶ cells were used for flow cytometry staining. All injections were carried out in the absence of anesthesia and analgesia, and mice were returned immediately to home cages following the procedure. The welfare of experimental animals was monitored regularly (typically immediately post procedure, then at least every 2-3 days). No unexpected adverse events were noted during the course of these experiments.

### Human Sample Preparation

Cryopreserved samples were thawed in a 37° C. water bath and vial contents transferred to a 15 mL Falcon tube. Pre-warmed defrost media (RPMI (Glutamax with HEPES) (Life Technologies (Thermo Fisher)), 5% human AB serum (Sigma), 20 nM TAPI-2 (Sigma), 50 U/mL Benzonase (Sigma) was added dropwise to 10 mL. Cells were rested in 4 mL resting media (RPMI with 10% human AB serum, 20 nM TAPI-2) for 1 hour at 37° C. 2 × 10⁶ cells were used for subsequent flow cytometry staining.

### Flow Cytometry

Mouse cells were surface stained with Fas PE (BD Biosciences, clone: Jo2), CD19 BUV395 (BD Biosciences, clone: 1D3), CD4 BUV395 (BD Biosciences, clone: GK1.5), CD4 PerCP-Cy5.5 (BD Biosciences, clone: RM4-5), GL7 AlexaFluor 488 (Biolegend, clone: GL7), CXCR5 BV421 (Biolegend, clone: L138D7), PD-1 PE-Cy7 (Biolegend, clone: RMP1-30), ICOS PE (eBioscience (Thermo Fisher), clone: 7E.17G9), CD45 BUV395 (BD Biosciences, clone: 30-F11), CD45RB APC (used in mouse panels in place of CD45RA, eBioscience (Thermo Fisher), clone: C363.16A) and DO11.10 TCR APC (eBioscience (Thermo Fisher), clone: KJ126) for 30 minutes at 4° C. Cells were fixed and permeabilised using the Foxp3/Transcription Factor Staining Buffer Set (eBioscience (Thermo Fisher)) and stained intracellularly with CD40L PE (BD Biosciences, clone: MR1) for 30 minutes at 4° C. For experiments involving Abatacept blockade in DO11 x RIP-mOVA mice, cells were stained with fixable viability dye eFluor 780 (eBioscience (Thermo Fisher)) in PBS for 10 minutes at 4° C. After washing once with PBS containing 2% fetal calf serum, samples were preincubated with purified anti-CD16/32 (BD Biosciences) for 5 minutes at 37° C. In FIG. 15,

mouse cells were preincubated with purified anti-CD16/32 for 5 minutes at 37° C. and stained with CXCR5 BV421 for 30 minutes at 37° C. Subsequently, an antibody cocktail containing CD3 BUV395 (BD Biosciences, clone: 145-2C11), CD4 PerCP-Cy5.5, CD45RB APC, CCR7 AlexaFluor 488 (Biolegend, clone: 4B12), PD-1 PE-Cy7, ICOS PE, CD25 PE-Cf594 (BD Biosciences, clone: PC61) and fixable viability dye eFluor 780 was added and cells were incubated for 30 minutes at 37° C.

Human cells were washed once in PBS and stained for 15 minutes at 37° C. with CCR7 BV605 (Biolegend, clone: G043H7) in Brilliant Stain Buffer (BD Biosciences). An antibody cocktail containing CD3 BUV395 (BD Biosciences, clone: SK7), CD4 PE-Cy7 (BD Biosciences, clone: SK3), CD25 BV421 (BD Biosciences, clone: M-A251), CD45RA PerCP-Cy5.5 (eBioscience (Thermo Fisher), clone: HI100), CD62L AlexaFluor 700 (Biolegend, clone: DREG-56), CD127 BV711 (BD Biosciences, clone: HIL-7R-M21), CXCR5 AlexaFluor 488 (BD Biosciences, clone: RF8B2), PD-1 PE (eBioscience (Thermo Fisher), clone: ebioJ105) and ICOS biotin (eBioscience (Thermo Fisher), clone: ISA-3) was subsequently added and cells were incubated for another 15 minutes at 4° C. Cells were then washed in PBS, streptavidin APC (BD Biosciences) was added to the residual volume and cells were incubated for 10 minutes at 4° C. Cells were resuspended in fixable viability dye eFluor 780 in PBS and incubated for 10 minutes at 4° C. before being washed in PBS twice. The marker CD62L was not considered in any of the downstream analysis. In FIG. 9, human cells were sequentially stained with CCR2 BV510 (Biolegend, clone: K036C2), CCR5 BUV737 (BD Biosciences, clone: 2D7) and CCR7 BV605 at 37° C. for 30, 20 and 15 minutes, respectively. Subsequently, an antibody cocktail containing CD3 BUV395, CD4 PE-Cy7, CXCR5 AlexaFluor 488, CD45RA PerCP-Cy5.5, HLA-DR BV785 (Biolegend, clone: L243), CD38 PE-Cf594 (BD Biosciences, HIT2), TIGIT BV421 (Biolegend, clone: A15153G) and BTLA BV650 (BD Biosciences, clone: J168-540) was added and cells were incubated for 15 minutes at 4° C. In FIG. 5, human cells were stained with CD3 BUV395, CD4 PECy7, CXCR5 AlexaFluor 488, CD45RA PerCP-Cy5.5, CXCR3 BV785 (Biolegend, clone: G025H7) and CCR6 APC-R700 (BD Biosciences, clone: 11A9) for 15 minutes at 4° C.

All data was acquired on a BD LSRFortessa (BD Biosciences). For manual analysis, data was analysed using FlowJo software version 10. For automated analysis, data was pre-gated on live CD3+ CD4+ cells in FlowJo, loaded into R using the Bioconductor package flowCore and underwent quality control using Bioconductor package FlowAI with standard configurations (Monaco, G., et al. (2016) Bioinformatics 32: 2473-2480). Low-quality events were removed and marker expression was transformed using arcsinh transformation using the Bioconductor package flowVS. CelICnn was run using a filter difference threshold of 0.5, maximum epochs of 100 and otherwise standard configurations. Filter specific cells were identified as cells having a filter response value in the upper 5% of the overall filter response. K-means clustering was performed using the CRAN package Stats, and optimal number of clusters were chosen using the Elbow method. Cluster information was added to fcs files using Bioconductor packages CytoML and flowWorkspace. The CRAN package Rtsne was used to compute t-SNE.

### Statistics and Predictive Modelling

Statistical analysis was performed using R v3.5.1 and Python v3.7. Two-sided Mann-Whitney U was used for comparison of two unpaired means. For comparison of paired means two-sided Wilcoxon signed-rank test was used. Comparison of more than two means was performed using two-sided ANOVA or Kruskal-Wallis test with Bonferroni correction. Equality of histograms in FIG. 9 and FIG. 15 was assessed using the Kolmogorov-Smirnov test. Normality was tested using Shapiro-Wilk test and homogeneity of variance was tested using Levene’s test. All measurements were taken from distinct samples. For boxplots, the black line indicates the median, the boxes represent first and third quartile and whiskers show minimum (first quartile - 1.5 * interquartile range) and maximum (third quartile + 1.5 * interquartile range). Principal component analysis was performed on scaled and centered data. Plots were produced using either CRAN packages ggplot2, ggpubr, ggsignif, RColourBrewer and scales in R or matplotlib and seaborn in Python. All predictive modelling was conducted using Python v3.7. Data cleaning and formatting was carried out using either CRAN packages plyr, stringr and tidyr in R or pandas and numpy in Python. The gradient boosting algorithm was implemented using sklearn.

### Results

### Abatacept Decreases Tfh in a Mouse Model of Diabetes

We examined adoptively transferred TCR transgenic T cells responding to a pancreasexpressed protein. Mice that express the DO11.10 TCR transgene in conjunction with its cognate antigen in pancreatic beta cells (DO11 x RIP-mOVA mice) develop spontaneous islet autoimmunity and diabetes with 100% penetrance. In these mice, islet-expressed OVA is presented to T cells in the pancreatic LN (PanLN), and this is associated with T cell differentiation to a Tfh phenotype.

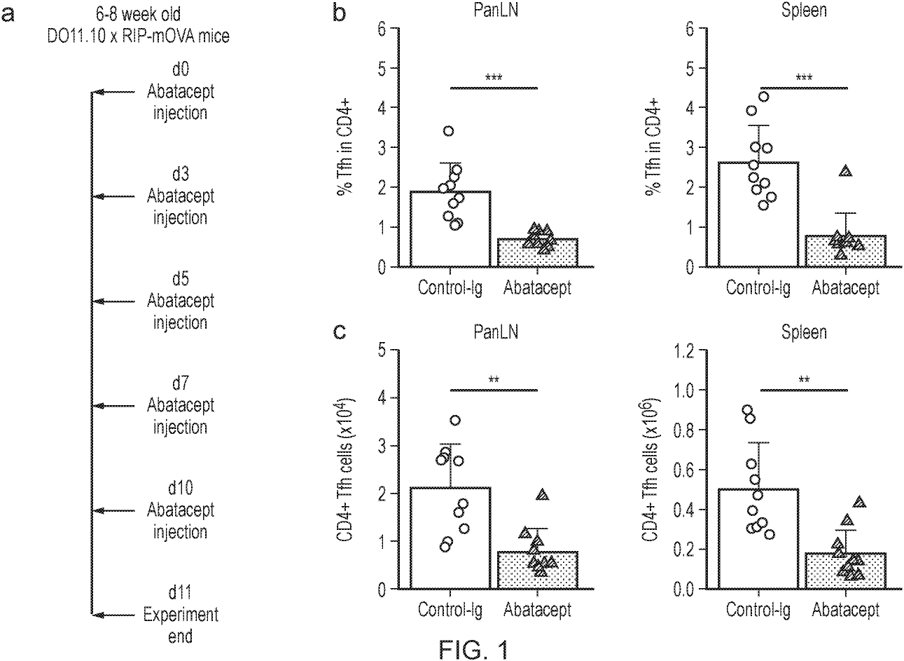

All mice manifest autoimmune islet infiltration by 5 weeks of age and we have established that CD28 costimulation is required for diabetes development (data not shown). To assess the impact of costimulation blockade on Tfh cells in the setting of an ongoing immune response to pancreatic autoantigen, we administered a short course of Abatacept to DO11 x RIP-mOVA mice (FIG. 1). The results of this experiment revealed a decrease in Tfh at the site of antigen presentation (PanLN) as well as the spleen (FIG. 1). Thus, even though T cell priming and Tfh differentiation were already underway prior to treatment, Abatacept was able to suppress the Tfh response.

### Abatacept Decreases Circulating Tfh in Type 1 Diabetes Patients

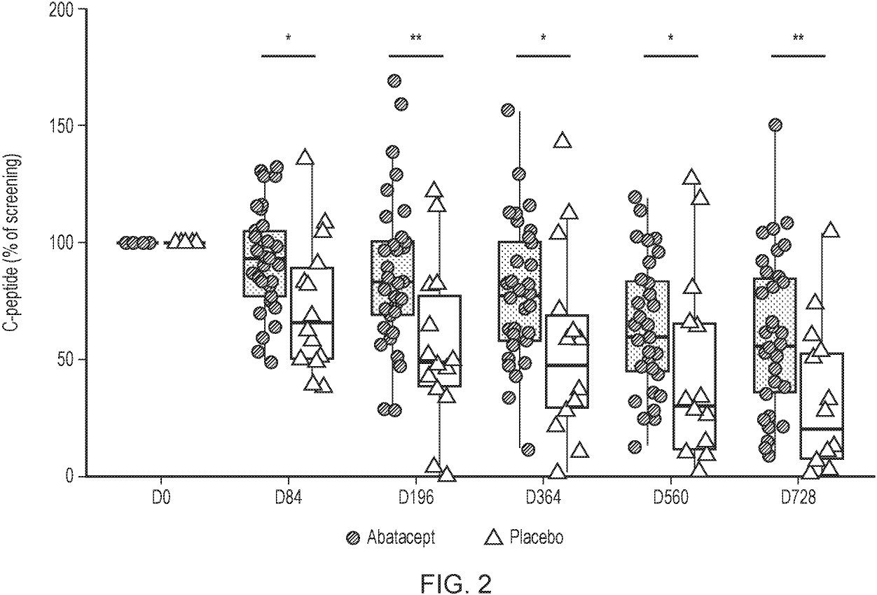

To assess whether Abatacept decreased circulating Tfh in humans with T1D, we obtained access to frozen samples from individuals with new onset T1D treated with Abatacept or placebo via Trialnet Study TN09 (NCT00505375). We were provided with samples from 36 Abatacept-treated individuals and 14 placebo-treated individuals, with 3 samples typically being available for each individual: baseline, and 1 and 2 years post treatment. Associated clinical data revealed a relative preservation of C-peptide in Abatacept-treated individuals compared with placebo-treated individuals (FIG. 2), in line with the original trial results from the entire cohort (Orban, T., et al. (2011) Lancet 378: 412-419).

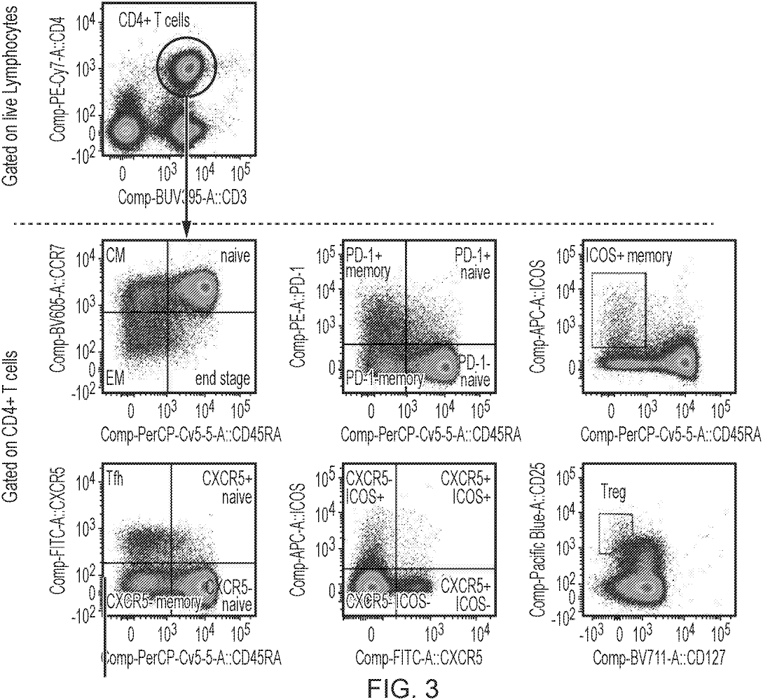

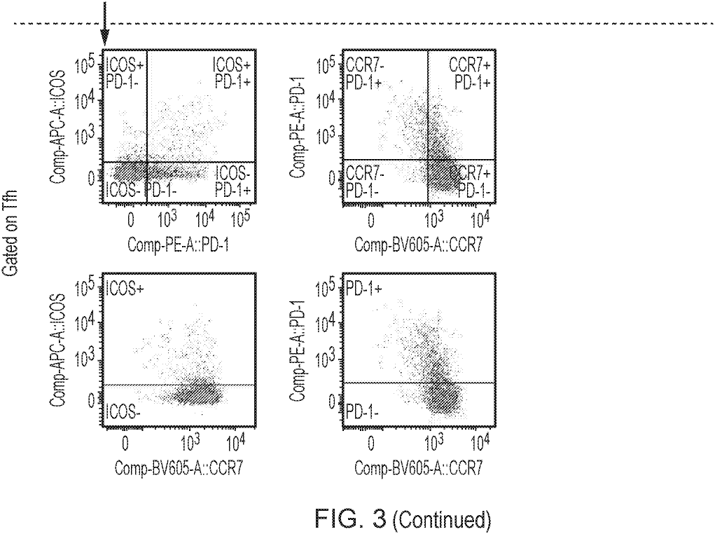

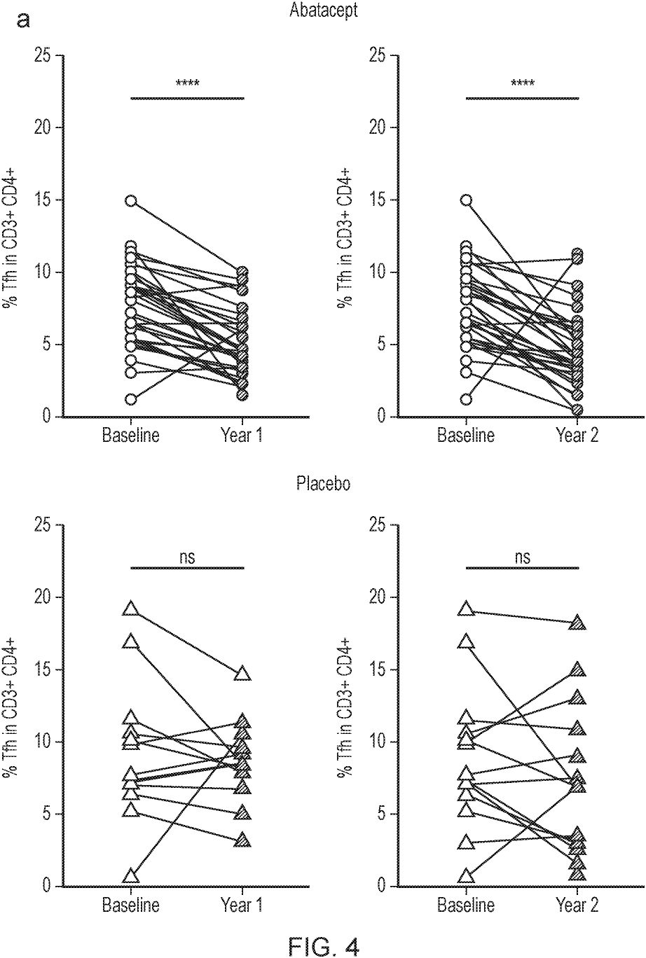

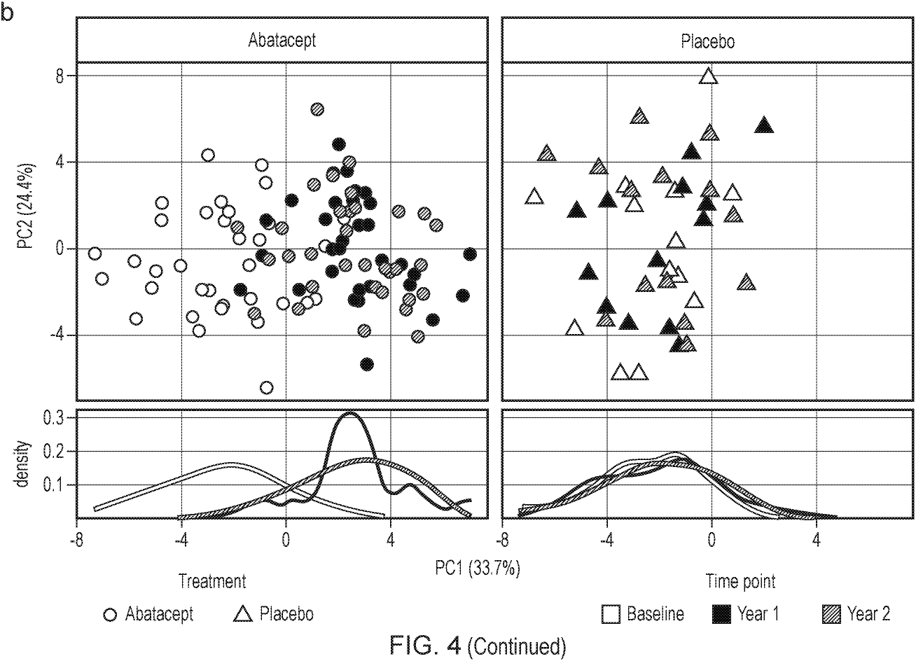

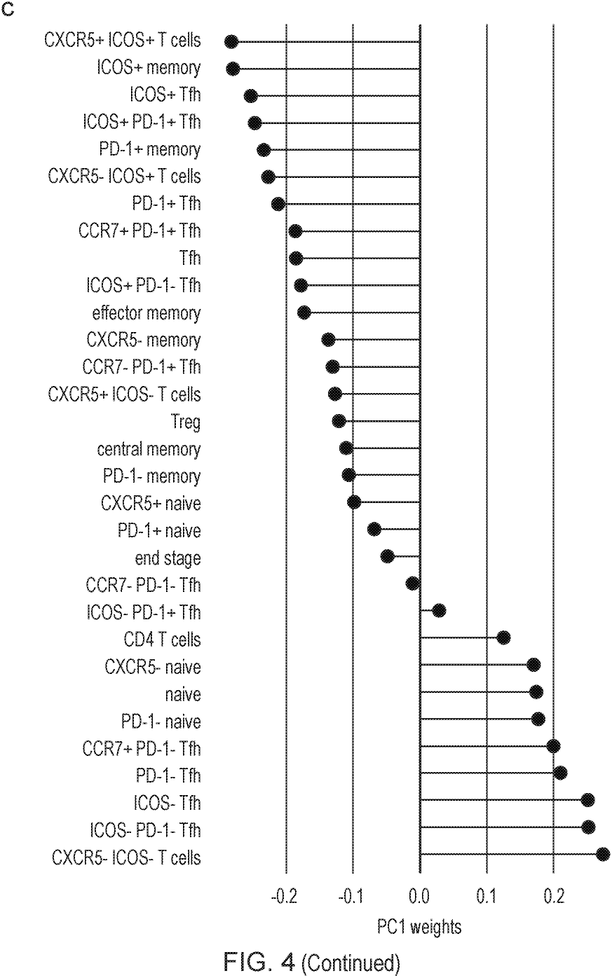

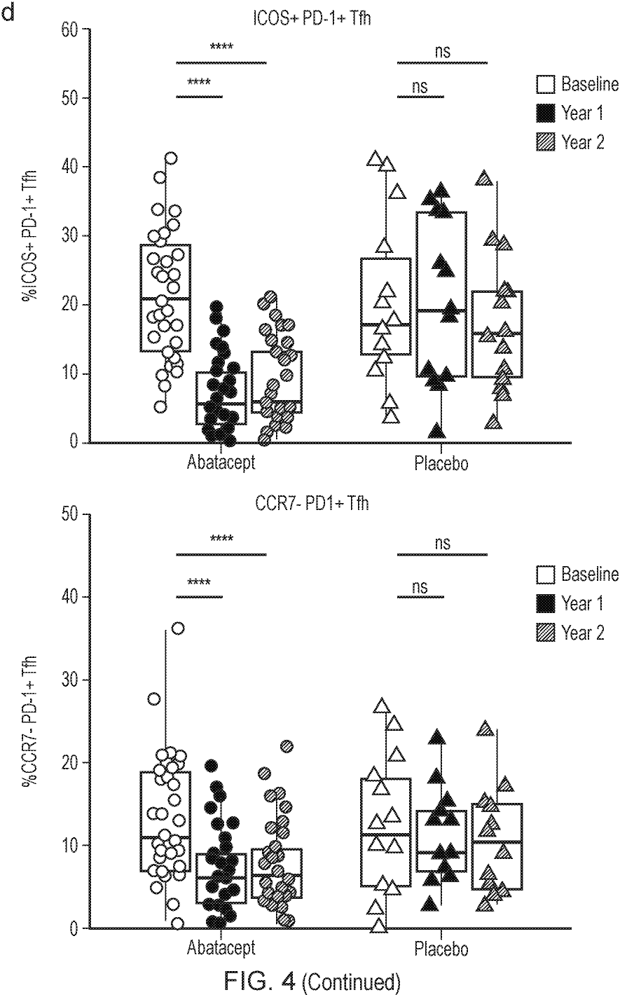

Samples were stained with a panel of T cell markers including ones associated with a Tfh phenotype (for gating strategy see FIG. 3). Since we previously showed that circulating CD4⁺CD45RA⁻CXCR5⁺ cells (Tfh) were overrepresented in humans with T1D (Kenefeck, R., et al. (2015) Journal of Clinical Investigation 125: 292-303), we first examined whether this population was Abatacept-sensitive. Our analysis revealed that Tfh were significantly decreased after Abatacept treatment at both 1 and 2 year timepoints, whereas this was not the case in the placebo-treated control group (FIG. 4). Principal component (PC) analysis of gated flow cytometry data revealed that the highest proportion of variance in this dataset is explained by Abatacept-induced changes, since treated samples are separated from untreated samples along PC1 for Abatacept treatment but not placebo treatment (FIG. 4). The major cell population contributing this separation was T cells expressing CXCR5 and ICOS (FIG. 4). CCR7^{lo}PD-1⁺CXCR5⁺ cells, previously identified as circulating Tfh precursors that correlate with disease activity in autoimmunity (He, J., et al. (2013) Immunity 39: 770-781), also contributed to PC1 and were decreased by Abatacept treatment (here called CCR7⁻PD-1⁺ Tfh) (FIG. 4). Graphed datapoints for the ICOS⁺PD-1⁺ Tfh and CCR7-PD-1⁺ Tfh populations are provided for illustrative purposes, and depict the Abatacept-induced change in cell frequency (FIG. 4d). To study the impact on Tfh subsets, additional trial samples were analysed with a panel incorporating CXCR3 and CCR628. The Abatacept-induced reduction of Tfh, and particularly ICOS⁺PD-1⁺Tfh, was corroborated, however there was no obvious skewing of CXCR3/CCR6-expressing subsets (FIG. 5).

### Additional Abatacept-Sensitive Populations in Type 1 Diabetes Revealed by CellCnn

Given the bias associated with manual gating, we tested whether unbiased analysis would also identify a change in Tfh-like cells following Abatacept treatment to independently validate the results of the PC analysis of manually gated flow cytometry data. We used the machine-learning algorithm CellCnn (Arvaniti, E. & Claassen, M. (2017) Nature communications 8: 14825), a representation learning approach using convolutional neural networks designed to identify rare cell subsets associated with disease status in a data-driven way. When samples are split into 2 groups (e.g. Abatacept versus placebo), this approach is able to establish marker expression profiles (filters) of individual cells whose frequency is associated with the assigned group.

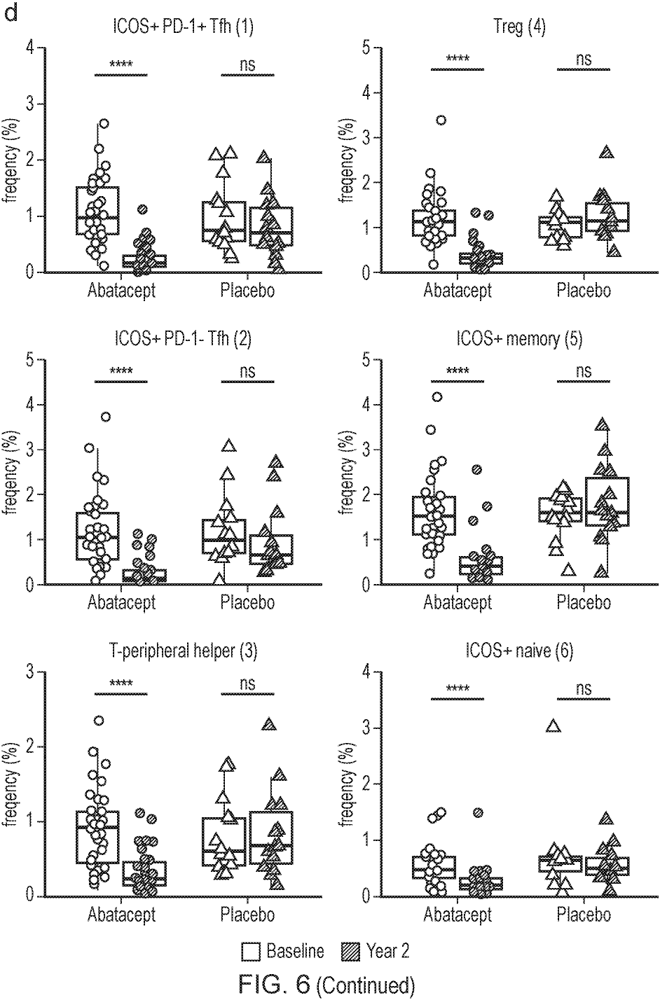

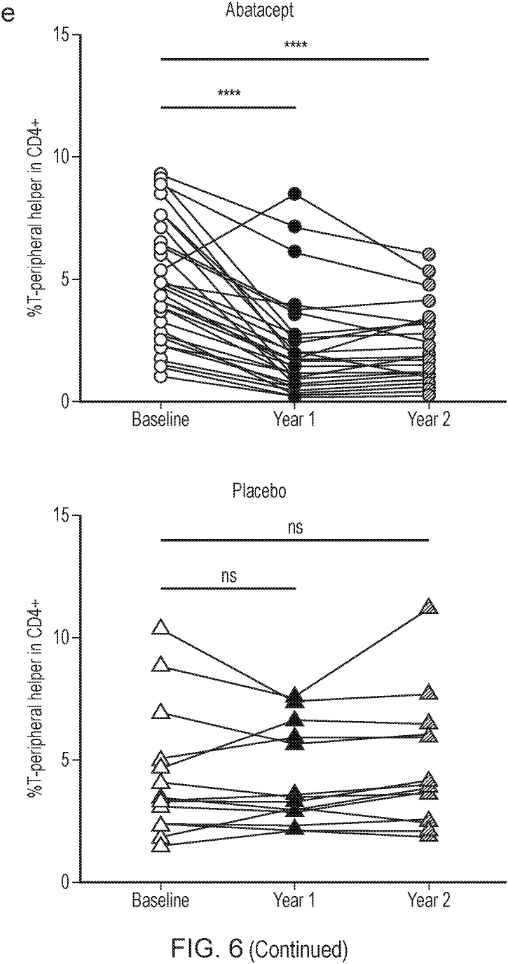

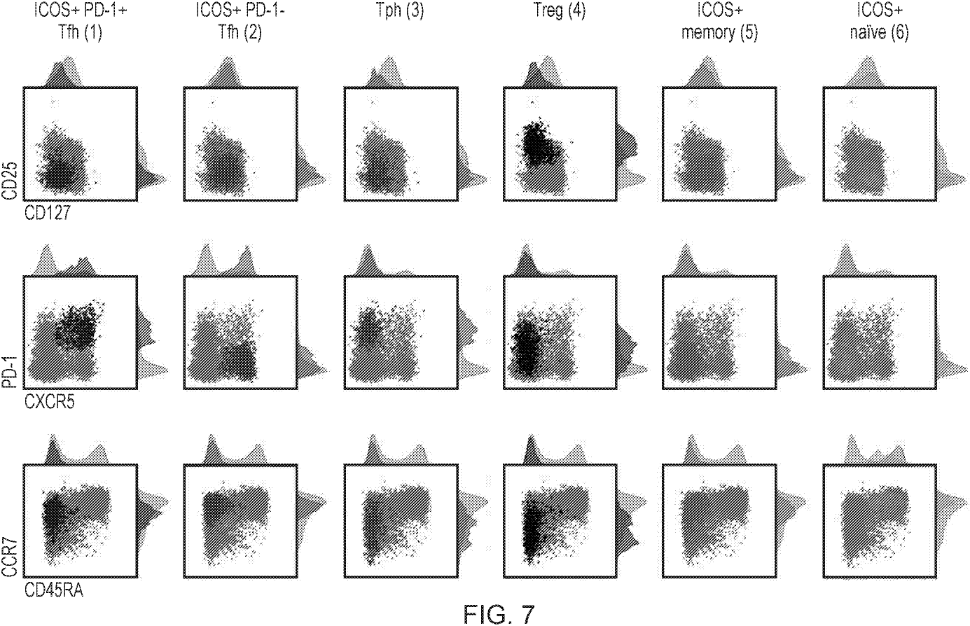

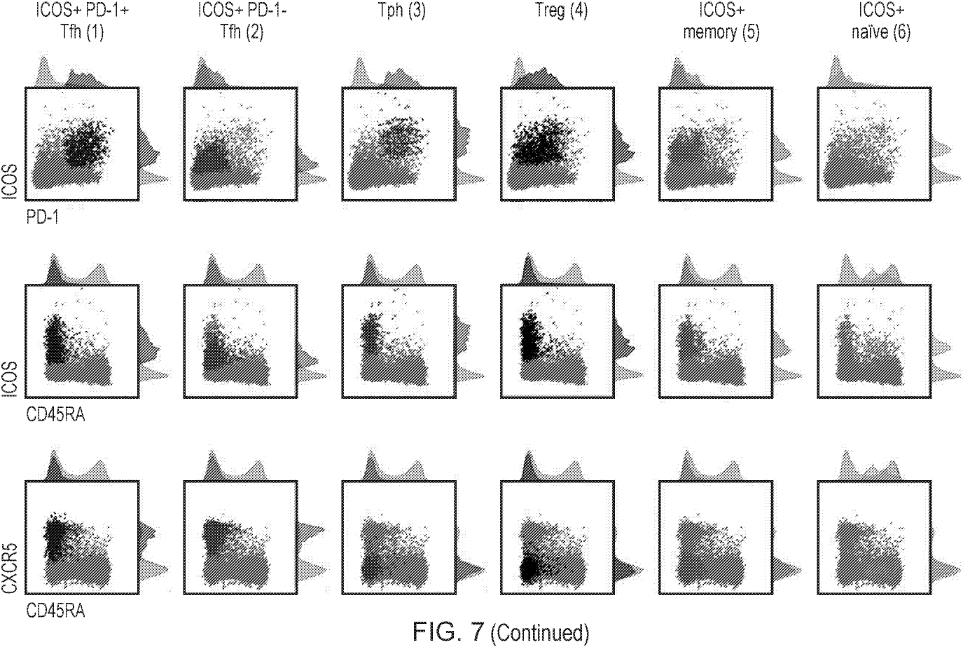

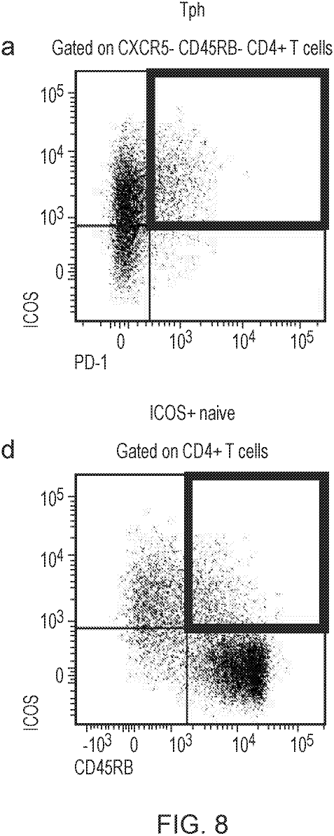

In our analysis, CellCnn identified a filter whose corresponding cells were present at high frequencies in all samples at baseline and in placebo treated samples, but were significantly reduced in Abatacept-treated samples after two years of treatment (FIG. 6a), indicating that this particular filter was associated with Abatacept-induced changes. Since filters detected by CellCnn do not necessarily represent a homogenous cell population, k-means clustering was applied to identify individual cell types affected by Abatacept treatment. In total 6 clusters were found (FIG. 6b) that showed distinct expression profiles of the selected markers. By overlaying these cell clusters on flow cytometry data (FIG. 6c, FIG. 7) we ascribed names to them that we believe reflect their identity, and assessed the change in the frequency of these populations in Abatacept or placebo treated individuals (FIG. 6d).

Consistent with our original manual gating approach, CellCnn identified both ICOS⁺PD-1⁺ Tfh (cluster 1) and ICOS⁺PD-1⁻ Tfh (cluster 2) to be decreased by Abatacept. A third cluster, comprising memory cells that lack CXCR5 but co-express ICOS and PD1 (cluster 3), was also identified as Abatacept responsive (FIG. 6d). This phenotype is reminiscent of the recently described T-peripheral helper cells (Tph) found in the rheumatoid joint (Rao, D.A., et al. (2017) Nature 542: 110-114). Manual gating of Tph confirmed a significant reduction in this population in people receiving Abatacept but not placebo at both year 1 and year 2 (FIG. 6e). CellCnn also identified Treg (cluster 4) to be Abatacept-sensitive, in addition to two other clusters characterised by ICOS expression (ICOS⁺ memory; cluster 5, ICOS⁺ naïve; cluster 6). Note that the term “naïve” is used as shorthand to reflect the fact that the cells in cluster 6 are CD45RA⁺, however their CD45RA expression level is slightly lower than bona fide naïve T cells (FIG. 6, cluster 6), suggesting they are antigen experienced. Thus machine-learning identified 2 Tfh populations and 4 additional populations to be Abatacept-sensitive, all of which expressed ICOS.

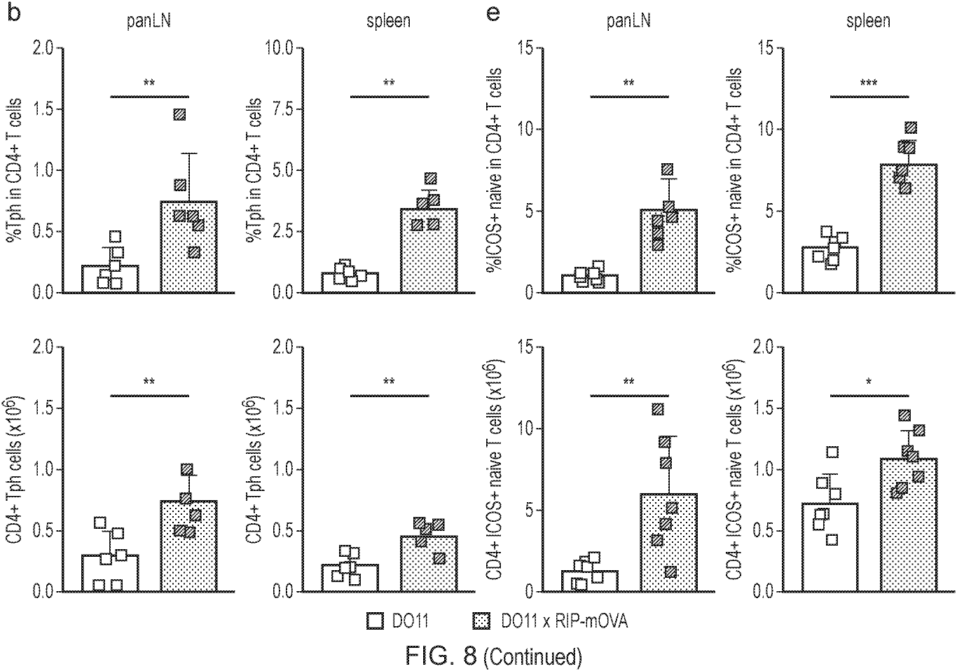

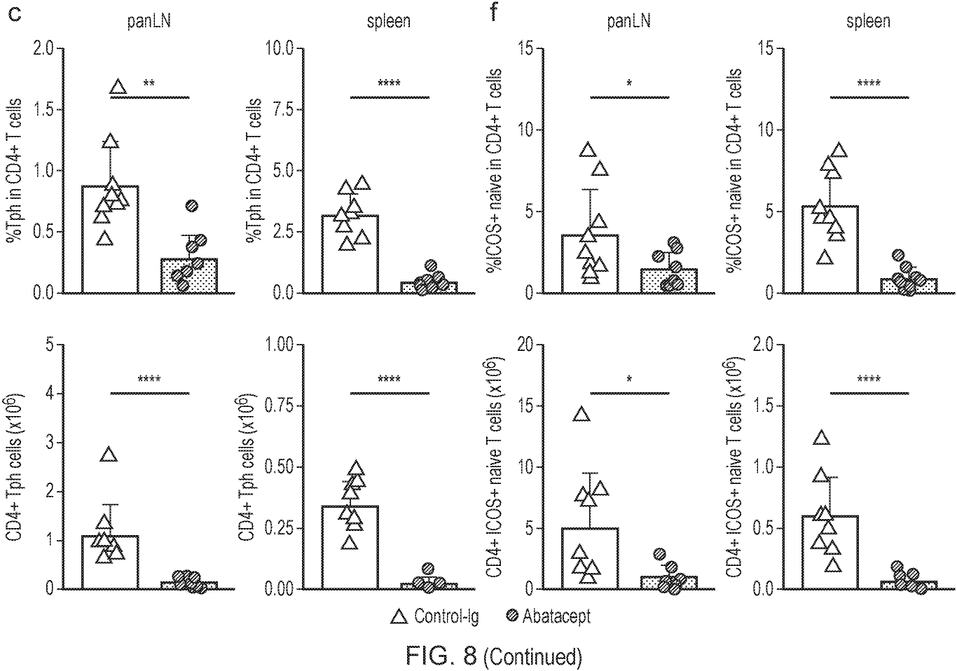

Since Tph have not previously been reported to be costimulation dependent, and ICOS⁺ naïve cells have not previously been described, we explored these populations further in our mouse model of diabetes. Cells with a “Tph” or “ICOS⁺naïve” phenotype could be identified in mice, were enriched in autoimmune animals, and were reduced following Abatacept treatment (FIG. 8). These murine data provide additional support for the costimulation sensitivity of these 2 populations.

To further explore the identity of the “Tph” population identified by CellCnn, additional trial samples were analysed. “Tph” cells were also decreased by Abatacept in this set of samples, and their expression of markers such as CCR5, CCR2, HLA-DR and CD38 was similar to that of Tph identified by standard gating, CXCR5⁻PD-1^{hi} (FIG. 9). Applying CellCnn to these data identified a cluster of cells expressing Tph markers to be costimulation-sensitive (FIG. 9).

### Baseline Tfh Phenotype Is Associated With Clinical Response to Abatacept

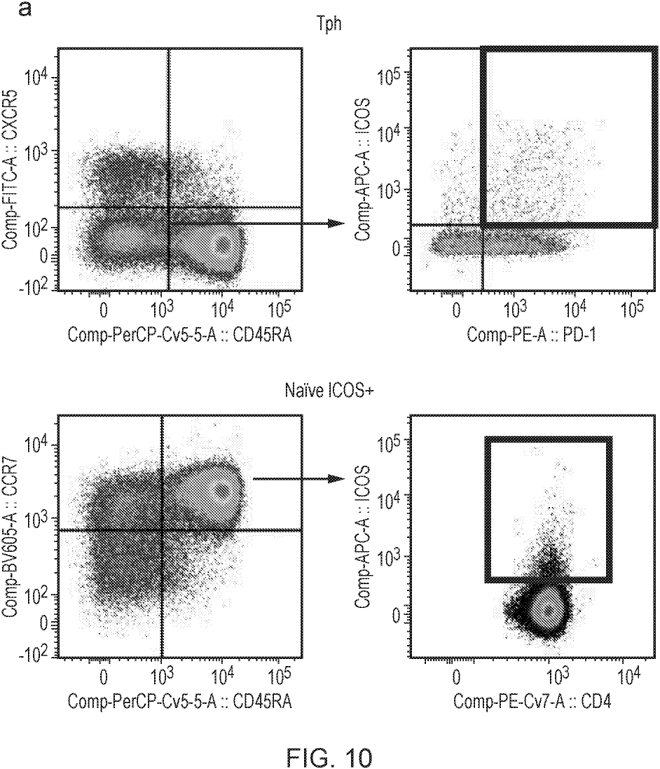

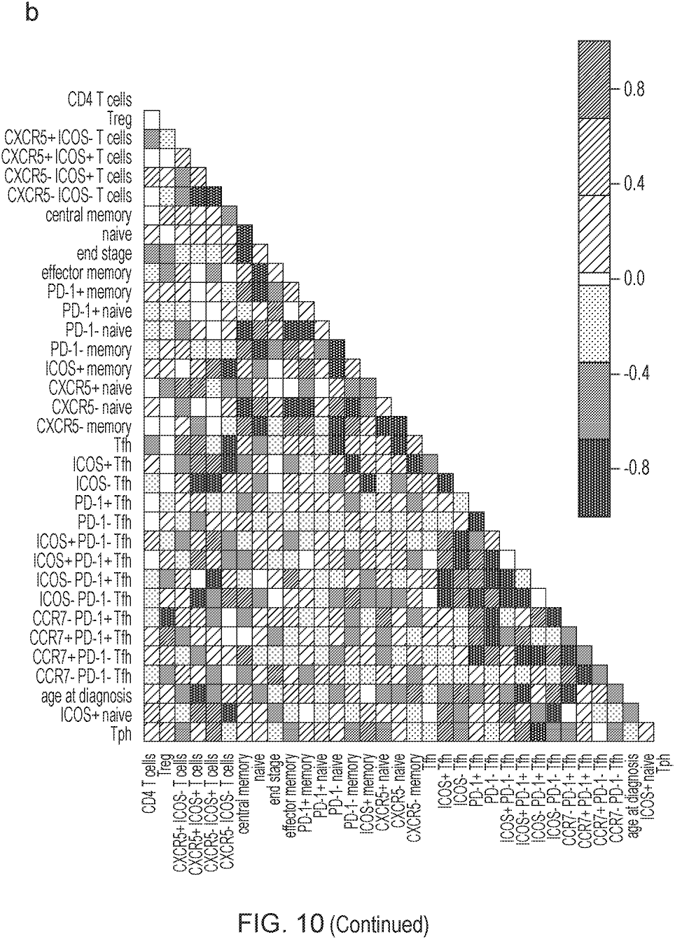

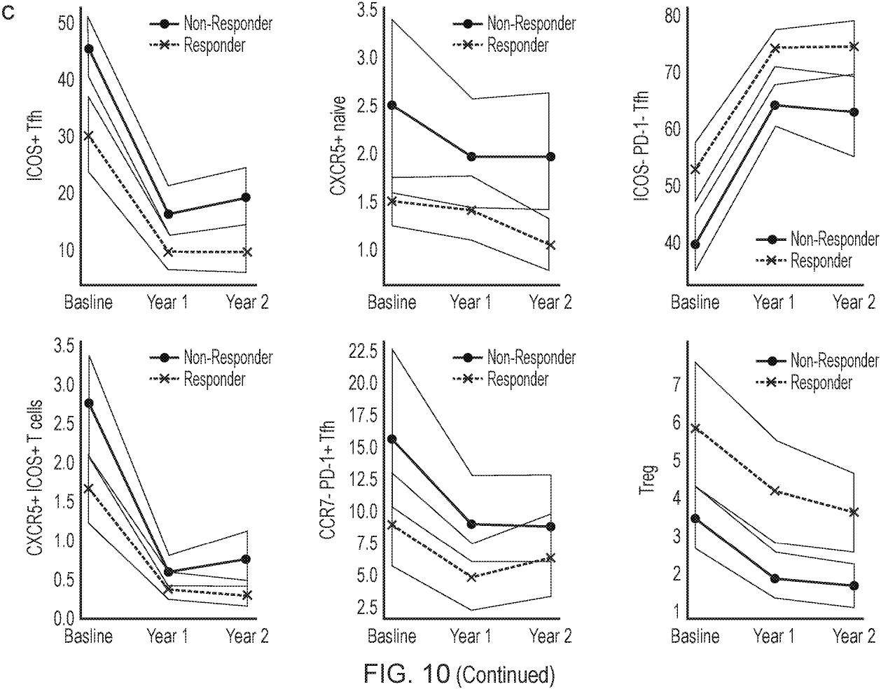

We next explored whether an individual’s clinical response following Abatacept treatment could be predicted from their T cell phenotype at baseline. Clinical response was assessed by relative C-peptide retention at the 2-year timepoint. Gated flow cytometry data were used, with a Tph gate and an ICOS⁺ naïve gate being added on the basis of their identification in the above analysis (FIG. 10). Age at diagnosis was also included since there is evidence that diagnosis at a young age is associated with a more rapid loss of beta cells.

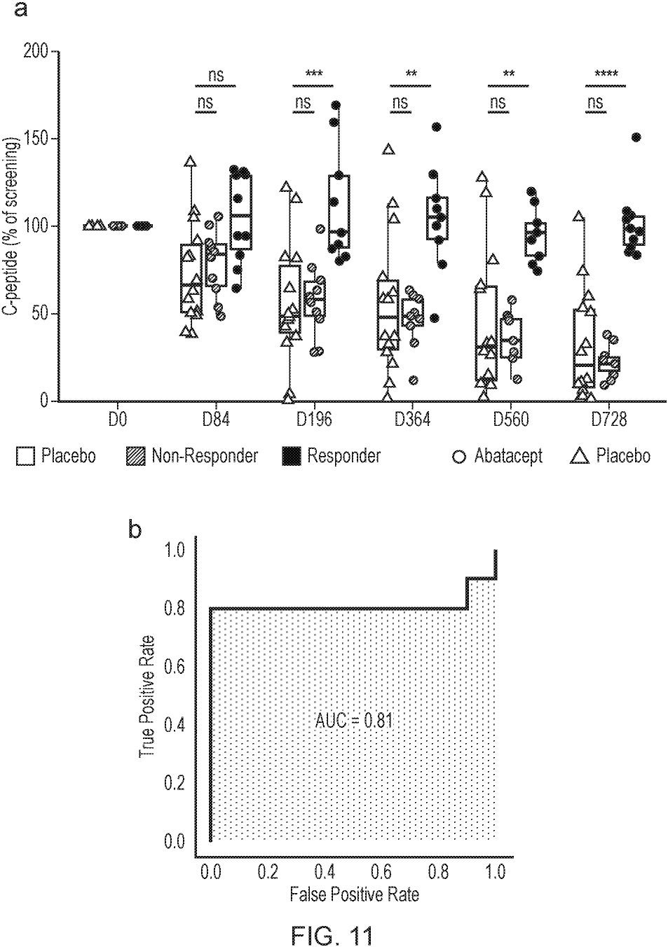

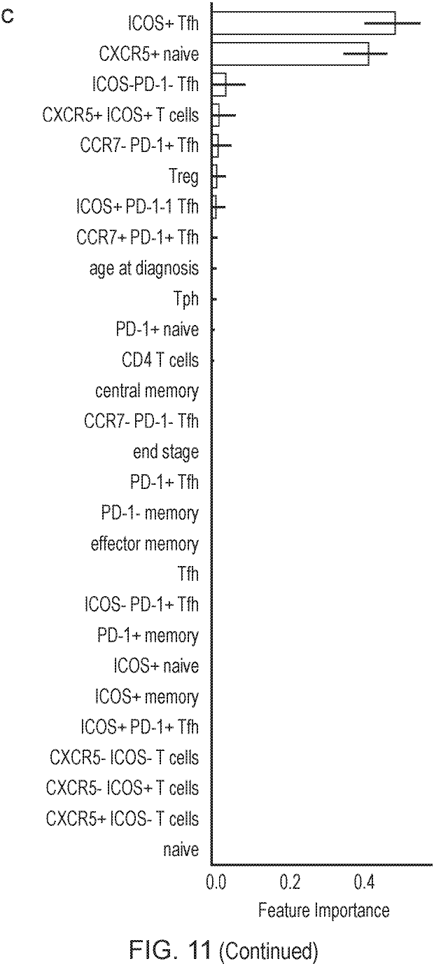

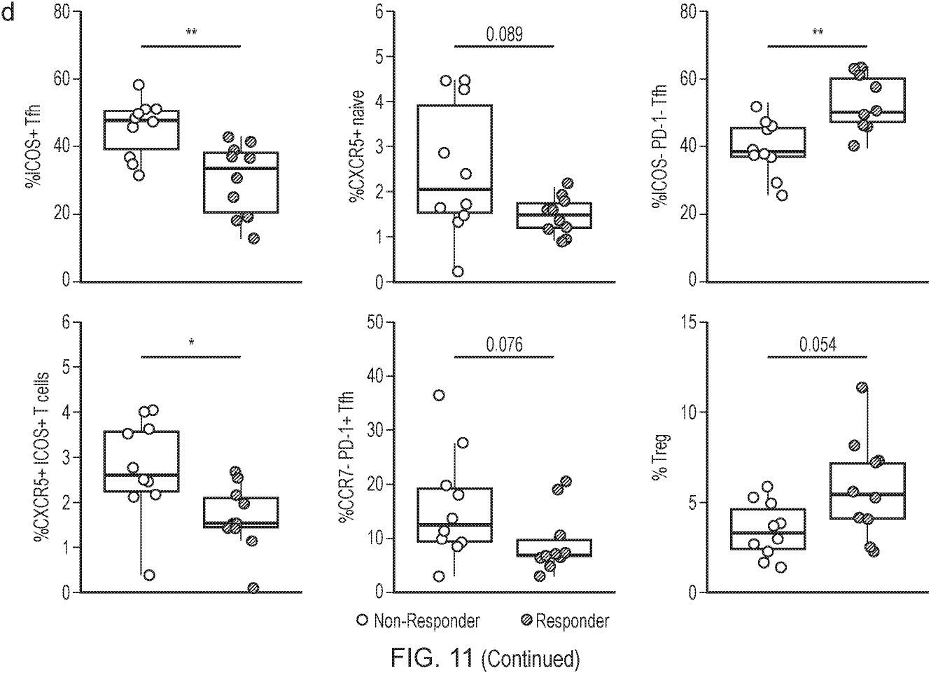

Within the Abatacept-treated subjects, the 10 with the best clinical response (responders) and the 10 with the poorest response (non-responders) (FIG. 11) were used to build a predictive model using gradient boosting (Breiman, L. (1997) Arcing the edge. Technical Report 486, Dept. Statistics, Univ. California, Berkeley. Available at www.stat.berkeley.edu; Friedman, J.H. (1999) Greedy Function Approximation: A Gradient Boosting Machine. Technical Report, Dept. Statistics, Stanford University). Pairwise correlation comparisons were conducted between features to identify and remove features that were highly correlated (Pearson correlation coefficient greater than 0.95), ensuring feature importance could be legitimately interpreted from our gradient boosting model (FIG. 10): where two features were shown to be highly correlated, the one least correlated with outcome was removed from the set of features used to build the predictive model. The gradient boosting model was constructed using nested leave-one-out cross validation. Each of the n patients was iteratively removed from the dataset and kept aside for testing purposes. The remaining n-1 baseline samples were used for model training and hyperparameter (learning rate, maximum depth and number of estimators) tuning using 3-fold cross validation. The optimal model from this training process was then used to make a prediction on the “left-out” sample, and feature weights were recorded.

We were able to predict response to Abatacept with 85% accuracy and an area under curve (AUC) of 0.81 (FIG. 11). The two features that emerged as being most important in predicting C-peptide retention following Abatacept treatment were ICOS⁺ Tfh (CD3⁺CD4⁺CD45RA⁻CXCR5⁺ICOS⁺) and CXCR5⁺ naïve cells (CD3⁺CD4⁺CXCR5⁺CD45RA⁺) (FIG. 11). Again, the term “naïve” is used as shorthand for CD45RA⁺, however cells in this gate have lower expression of CD45RA than naïve T cells (see “CXCR5⁺ naïve” quadrant in FIG. 3). ICOS-PD-1⁻ Tfh also contribute to predictive power in this model, with opposing directionality to ICOS+ Tfh as expected (FIG. 11). The CCR7^{lo}PD-1⁺CXCR5⁺ cells discussed above are also identified in the model (CCR7⁻PD-1⁺ Tfh) (FIG. 11). Grouped time-series plots illustrate the dynamic change in the frequencies of these cell populations over time (FIG. 10), illustrating that responder and non-responder populations are broadly non-overlapping both before and during Abatacept treatment. Note that only baseline data were used to generate the model, avoiding the caveat that Abatacept treatment directly alters the frequencies of some of these populations.

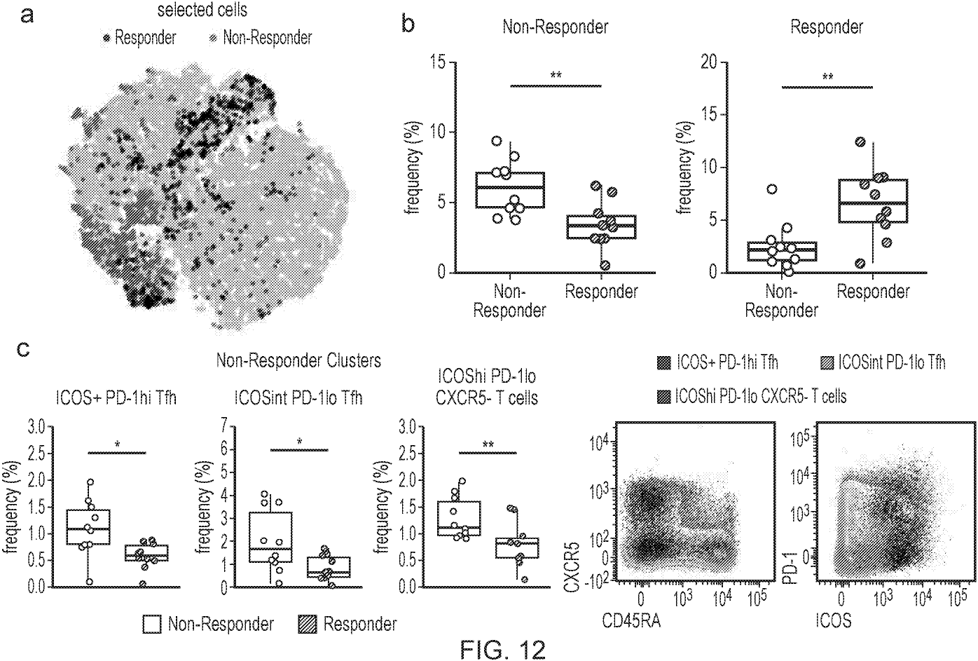

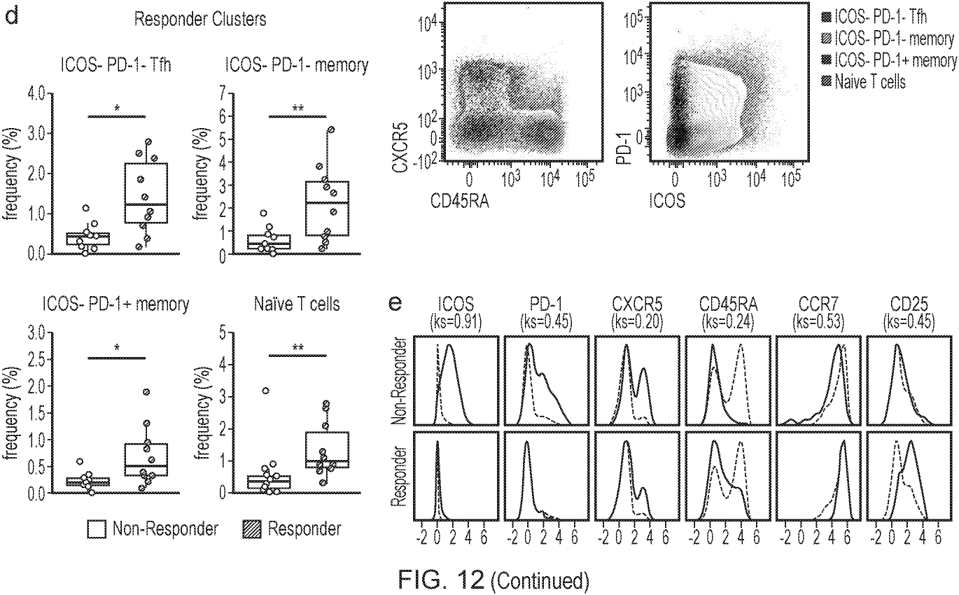

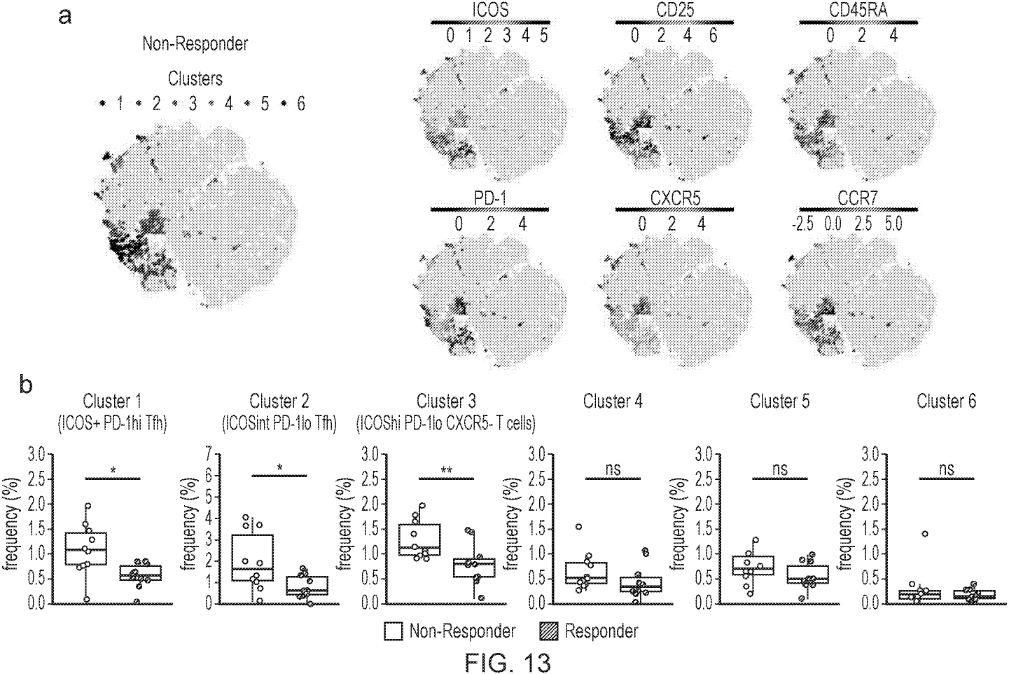

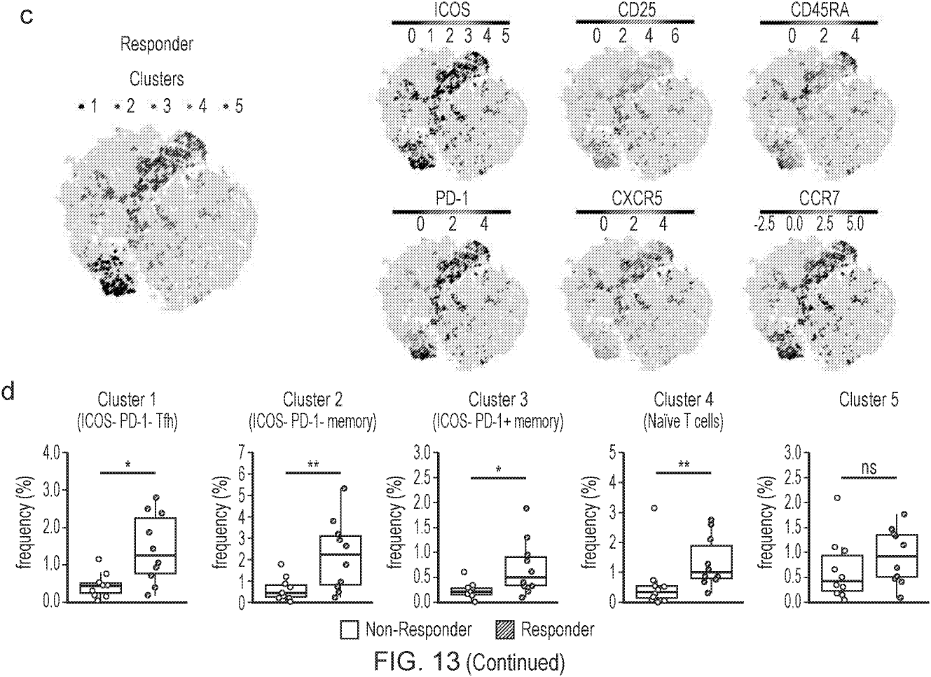

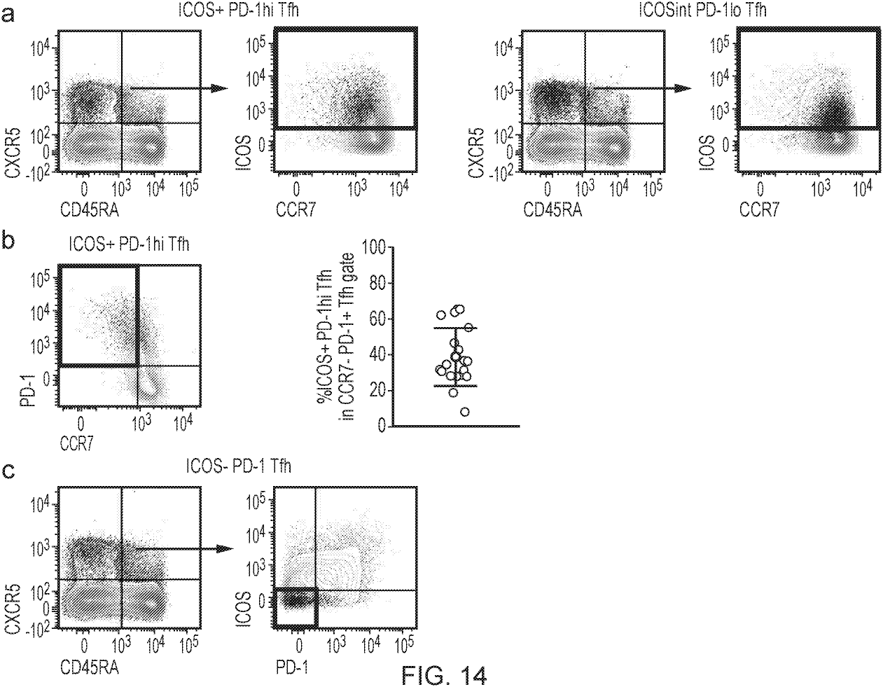

As an independent approach, we were interested in whether data-driven analysis would detect similar cell subsets at baseline that differed between individuals who went on to make good or poor clinical responses following Abatacept therapy. Using CellCnn we were able to identify two filters, one of which shows higher frequencies of corresponding cells in samples from the 10 individuals exhibiting the poorest clinical response, while the other exhibits an inverse relationship, leading us to label these filters as “Non-Responder” and “Responder”, respectively (FIG. 12). In the non-responder filter, k-means clustering revealed 3 statistically significant T cell clusters; ICOS⁺PD-1^{hi} Tfh, ICOS^{int}PD-1^{lo} Tfh and ICOS^{hip}D-1^{lo}CXCR5⁻ T cells (FIG. 12, FIG. 13). The first 2 of these provide independent support for the predictive power of the ICOS⁺ Tfh population identified in our gradient boosting model. Indeed, cells identified by CellCnn in those clusters overlaid the manual gates used for the predictive model (FIG. 14). ICOS⁺PD-1^{hi} Tfh partially encompasses the CCR7⁻PD1⁺ Tfh population also identified by the model (FIG. 14b). Conversely, the clusters identified in the filter found for responder patients were dominated by ICOS⁻ cell populations, including ICOS⁻ PD-1⁻ Tfh, ICOS⁻PD-1⁻ memory cells, ICOS-PD-1⁺ memory cells and naïve T cells (FIG. 12, FIG. 13, FIG. 14).

The difference in ICOS expression between patients that go on to be Abatacept responders versus non-responders is clear from a combined analysis of all cells contributing to clusters identified in both filters (FIG. 12). Analysis of Abatacept-treated pre-diabetic mice showed that an analogous staining panel could be used to build a predictive model of clinical response with 84% accuracy and an AUC of 0.83 (FIG. 15). CellCnn identified filters that were enriched in pre-diabetic mice that went on to be responders or non-responders, with ICOS being expressed at higher levels in the cells within the non-responder clusters (FIG. 15).

### Conclusions

We show here that in both mice and humans experiencing ongoing autoimmune responses, costimulation blockade with Abatacept reduced Tfh frequencies. Importantly, we identified several new Abatacept-sensitive populations, including a population resembling Tph cells (ICOS⁺PD-1⁺CXCR5⁻) which are thought to provide T cell help to B cells in the rheumatoid synovium. Emerging data suggest these cells are expanded in children with islet autoantibodies who go on to develop diabetes (Ekman, I., et al. (2019) Diabetologia 62: 1681-1688), and are associated with disease activity in SLE (Bocharnikov, A.V., et al. (2019) JCI insight 4 e130062) and RA (Zhang, F., et al. (2019). Nat Immunol. 20: 928-942; Fortea-Gordo, P., et al. (2019) Rheumatology (Oxford) 58: 1662-1673), suggesting insights into their drug sensitivity could have broad applicability.

Furthermore, we found that cells resembling the circulating Tfh precursors (CXCR5⁺CCR7^{lo}PD-1⁺) also exhibit Abatacept sensitivity. The appreciation that costimulation blockade can target Tfh precursors that rapidly differentiate into mature Tfh upon antigen encounter, provides further mechanistic insight into this therapy.

Our work also uncovered a novel population of Abatacept-sensitive T cells expressing CD45RA and intermediate levels of ICOS (termed “ICOS⁺ naïve” in FIG. 6). These could conceivably represent T cells that have undergone recent activation and not yet fully lost CD45RA, or alternatively revertants that have lost, then re-expressed, this marker. Evidence that memory CD4 T cells can revert to expressing CD45R isoforms associated with the naïve state first emerged from rat and mouse models, where it was established that revertant T cells retained the capacity to provide B cell help (Bell, E.B., et al. (2001) Eur J Immunol 31: 1685-1695).

Having established that the frequency and phenotype of Tfh could serve as a biomarker of costimulation blockade, we sought to explore the predictive value of Tfh analysis. We used gradient boosting, an ensemble machine learning method, on gated flow cytometry outputs from pre-treatment samples, and were able to build a predictive model of Abatacept sensitivity that could assign the clinical response at year 2 with 85% accuracy. The model has strong predictive power and sheds light on a handful of T cell populations whose collective frequencies appear to inform the clinical response to Abatacept. Chief among these is the ICOS⁺Tfh population, for which higher frequencies are associated with a poor clinical response. Reciprocally, ICOS⁻PD-1⁻ Tfh also contribute to the model, with a higher frequency being associated with a better clinical response following Abatacept treatment.

Using CellCnn we were able to provide independent corroboration for key aspects of our model. Notably this approach confirmed that a poor clinical response was associated with higher frequencies of ICOS⁺ Tfh at baseline. These were divided by the clustering algorithm into those with either high or low PD-1 (cluster 1 and cluster 2 respectively; FIG. 13). Conversely, a good clinical response was confirmed to be associated with higher frequencies of ICOS⁻PD-1⁻ Tfh (FIG. 13). In addition to identifying the importance of ICOS expression in Tfh, this analysis also revealed an effect of ICOS expression on CXCR5⁻ cells. Thus, ICOS appears to be the most discerning cellular marker associated with preservation of beta-cell function following Abatacept treatment as assessed by two independent analysis techniques.

In our mouse model of autoimmune diabetes, because disease develops over weeks rather than years it is not possible to distinguish between Stage 1 and Stage 2 diabetes. However, we are able to identify mice with abnormal glucose homeostasis that do not yet have overt diabetes (pre-diabetic). We have therefore tested our biomarker approach in these pre-diabetic animals, providing evidence that it can be informative prior to the development of overt disease (FIG. 15).

Robust predictive markers of responsiveness to Abatacept are currently lacking, although there are suggestions that individuals with greater inflammatory activity exhibit a better clinical response (Cabrera, S.M., et al. (2018) Diabetologia 61: 2356-2370). A recent study using whole blood RNASeq detected changes in expression of B cell genes that were associated with clinical response in subjects with T1D treated with Abatacept (Linsley, P.S et al. (2019) JCI insight 4 e126136), however these were not apparent until 84 days post treatment initiation. Our report is the first to suggest that baseline (i.e. pre-treatment) Tfh phenotypes have the potential to predict clinical response to an immunotherapy.

Overall, both the predictive model and the CellCnn algorithm evidenced that analysis of Tfh markers in baseline blood samples could predict clinical response following Abatacept immunotherapy.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### ASPECTS OF THE INVENTION

Aspects of the invention are defined by the following numbered paragraphs:

1. A method for identifying a subject with an autoimmune or inflammatory disease who is suitable for treatment with costimulation blockade therapy, the method comprising determining the profile of B helper T cells in a sample from the subject.

2. A method for predicting or determining whether a subject with an autoimmune or inflammatory disease will respond to treatment with costimulation blockade therapy, the method comprising determining the profile of B helper T cells in a sample from the subject.

3. A method of treating or preventing an autoimmune or inflammatory disease in a subject which comprises treating a subject with or at risk of developing an autoimmune or inflammatory disease with costimulation blockade therapy, wherein the subject has been identified as suitable for treatment with the costimulation blockade therapy by determining the profile of B helper T cells in a sample from the subject.

4. The method according to any one of the preceding paragraphs, wherein the profile of B helper T cells is determined using at least one marker on CD4⁺ T cells selected from the group consisting of CXCR5, ICOS, PD-1, CD45RA, CD127 and/or CD25.

5. The method according to any one of the preceding paragraphs, wherein the frequency of at least one B helper T cell phenotype is determined, optionally wherein the at least one B helper T cell phenotype is selected from the group consisting of ICOS -PD-1⁻ follicular helper T cells (Tfh), ICOS⁺ Tfh, CCR7⁻PD-1⁺ Tfh, CXCR5⁺ICOS⁺ T cells, CXCR5⁻ICOS⁺ T cells, ICOS⁺PD-1^{high} Tfh, ICOS-PD-1- memory T cells, ICOS-PD-1⁺ memory T cells and CXCR5⁺ naïve T cells.

6. The method according to paragraph 5, wherein the frequency of at least three B helper T cell phenotypes is determined.

7. The method according to paragraph 6, wherein the at least three B helper T cell phenotypes are ICOS -PD-1⁻ Tfh, ICOS⁺ Tfh and CCR7⁻PD-1⁺ Tfh.

8. The method according to any one of the preceding paragraphs, further wherein the frequency of at least one of naïve T cells and/or regulatory T cells (Treg) is determined in the sample from the subject.

9. The method according to any one of the preceding paragraphs, wherein the profile of B helper T cells in the sample is compared to a reference frequency, wherein the reference frequency is selected from:
(a) a population of subjects who are non-responsive to the costimulation blockade therapy; and/or
(b) a population of subjects who are responsive to the costimulation blockade therapy.

10. The method according to paragraph 9, wherein the reference frequency is from a population of subjects who are non-responsive to the costimulation blockade therapy and wherein:
(a) a higher frequency of ICOS⁻PD-1⁻ Tfh;
(b) a lower frequency of ICOS⁺ Tfh;
(c) a lower frequency of CCR7⁻PD-1⁺ Tfh;
(d) a lower frequency of CXCR5⁺ICOS⁺ T cells;
(e) a lower frequency of CXCR5⁻ICOS⁺ T cells;
(f) a lower frequency of ICOS⁺PD-1^{high} Tfh;
(g) a higher frequency of ICOS⁻PD-1⁻ memory T cells;
(h) a higher frequency of ICOS-PD-1⁺ memory T cells;
(i) a lower frequency of CXCR5⁺ naïve T cells;
(j) a higher frequency of naïve T cells; and/or
(k) a higher frequency of Treg,
in comparison to a reference frequency is indicative of response to the treatment.

11. The method according to any one of the preceding paragraphs, wherein the method comprises using at least one predictive modelling approach to identify the subject suitable for treatment with costimulation blockade therapy or to predict or determine whether the subject will respond to treatment with costimulation blockade therapy.

12. The method according to paragraph 11, wherein the at least one predictive modelling approach is gradient boosting, random forests, support vector machines and/or logistic regression.

13. The method according to paragraph 11 or paragraph 12, wherein populations of subjects grouped according to clinical response are used as inputs to the predictive modelling approach.

14. A method of treating or preventing an autoimmune or inflammatory disease in a subject, wherein the method comprises the following steps:
(a) identifying or determining a subject with or at risk of developing an autoimmune disease who is suitable for treatment with costimulation blockade therapy by the method according to any one of paragraphs 1, 2 or 4-13; and
(b) treating the subject with costimulation blockade therapy.

15. A method of treating or preventing an autoimmune or inflammatory disease in a subject which comprises treating a subject with or at risk of developing an autoimmune or inflammatory disease with costimulation blockade therapy, which subject has been identified or determined as suitable for treatment with costimulation blockade therapy by the method according to any one of paragraphs 1, 2 or 4-13.

16. A costimulation blockade therapy for use in a method of treatment or prevention of an autoimmune or inflammatory disease in a subject, the method comprising:
(a) identifying or determining a subject with or at risk of developing an autoimmune or inflammatory disease who is suitable for treatment with costimulation blockade therapy by the method according to any one of paragraphs 1, 2 or 4-13; and
(b) treating the subject with costimulation blockade therapy.

17. A costimulation blockade therapy for use in treating or preventing an autoimmune or inflammatory disease in a subject, wherein the subject has been identified as suitable for treatment with the costimulation blockade therapy by determining the profile of B helper T cells in a sample from the subject.

18. The costimulation blockade therapy for use according to paragraph 17, wherein the profile of B helper T cells is determined using at least one marker on CD4+ T cells selected from the group consisting of CXCR5, ICOS, PD-1, CD45RA, CD127 and/or CD25.

19. The costimulation blockade therapy for use according to paragraph 17 or paragraph 18, wherein the frequency of at least one B helper T cell phenotype is determined, wherein the at least one B helper T cell phenotype is selected from the group consisting of ICOS -PD-1⁻ follicular helper T cells (Tfh), ICOS⁺ Tfh, CCR7⁻PD1⁺ Tfh, CXCR5⁺ICOS⁺ T cells, CXCR5-ICOS⁺ T cells, ICOS⁺PD-1^{high} Tfh, ICOS-PD-1- memory T cells, ICOS-PD-1⁺ memory T cells and CXCR5⁺ naïve T cells.

20. The costimulation blockade therapy for use according to paragraph 19, wherein the frequency of at least three B helper T cell phenotypes is determined.

21. The costimulation blockade therapy for use according to paragraph 20, wherein the at least three B helper T cell phenotypes are ICOS -PD-1⁻ Tfh, ICOS⁺ Tfh and CCR7⁻PD-1⁺ Tfh.

22. The costimulation blockade therapy for use according to any one of paragraphs 17-21, further wherein the frequency of at least one of naïve T cells and/or regulatory T cells (Treg) is determined in the sample from the subject.

23. The costimulation blockade therapy for use according to any one of paragraphs 17-22, wherein the profile of B helper T cells in the sample is compared to a reference frequency, wherein the reference frequency is from:
(a) a population of subjects who are non-responsive to the costimulation blockade therapy; and/or
(b) a population of subjects who are responsive to the costimulation blockade therapy.

24. The costimulation blockade therapy for use according to paragraph 23, wherein the reference frequency is from a population of subjects who are non-responsive to the costimulation blockade therapy and wherein:
(a) a higher frequency of ICOS⁻PD-1⁻ Tfh;
(b) a lower frequency of ICOS⁺ Tfh;
(c) a lower frequency of CCR7⁻PD-1⁺ Tfh;
(d) a lower frequency of CXCR5⁺ICOS⁺ T cells;
(e) a lower frequency of CXCR5⁻ICOS⁺ T cells;
(f) a lower frequency of ICOS⁺PD-1^{high} Tfh;
(g) a higher frequency of ICOS⁻PD-1⁻ memory T cells;
(h) a higher frequency of ICOS-PD-1⁺ memory T cells;
(i) a lower frequency of CXCR5⁺ naïve T cells;
(j) a higher frequency of naïve T cells; and/or
(k) a higher frequency of Treg,
in comparison to a reference frequency is indicative of response to the treatment.

25. The costimulation blockade therapy for use according to any one of paragraphs 17-26, wherein the subject is identified as suitable for treatment with costimulation blockade therapy using at least one predictive modelling approach.

26. The costimulation blockade therapy for use according to paragraph 25, wherein the at least one predictive modelling approach is gradient boosting, random forests, support vector machines and/or logistic regression.

27. The costimulation blockade therapy for use according to paragraph 25 or paragraph 26, wherein populations of subjects grouped according to clinical response are used as inputs to the predictive modelling approach.

28. A costimulation blockade therapy for use in treating or preventing an autoimmune disease in a subject, which subject has been identified or determined as suitable for treatment with costimulation blockade therapy by the method according to any one of paragraphs 1,2 or 4-13.

29. The method or costimulation blockade therapy for use according to any one of the preceding paragraphs, wherein the autoimmune or inflammatory disease is selected from the group consisting of type 1 diabetes, rheumatoid arthritis, psoriatic arthritis, juvenile idiopathic arthritis, Sjogren’s syndrome, Graves’s Disease, Myasthenia Gravis, inflammatory vascular diseases, glomerulonephritis, diabetic nephropathy and systemic lupus erythematosus including systemic lupus erythematosus arthritis.

30. The method or costimulation blockade therapy for use according to any one of the preceding paragraphs, wherein the autoimmune disease is type 1 diabetes.

31. The method or costimulation blockade therapy for use according to any one of the preceding paragraphs, wherein the sample is a blood sample.

32. The method or costimulation blockade therapy for use according to any one of the preceding paragraphs, wherein the costimulation blockade therapy is CD28 costimulation blockade therapy.

33. The method or costimulation blockade therapy for use according to paragraph 32, wherein the CD28 costimulation blockade therapy is selected from the group consisting of a CTLA-4-lg fusion protein, such as Abatacept, Belatacept and MEDI5265; an anti-CD28 antagonist antibody, such as lulizumab; and FR104.

34. The method or costimulation blockade therapy for use according to any one of the preceding paragraphs, wherein the subject is a human.

35. The method or costimulation blockade therapy for use according to any one of the preceding paragraphs, wherein the profile of B helper T cells is determined by flow cytometry.

36. The method or costimulation blockade therapy for use according to any one of the preceding paragraphs, wherein determining the profile of B helper T cells in the sample is carried out:
(a) prior to the onset of symptoms of the autoimmune or inflammatory disease;
(b) while the subject is showing symptoms of the autoimmune or inflammatory disease;
(c) prior to the use of costimulation blockade therapy to treat or prevent the autoimmune or inflammatory disease; and/or
(d) during and/or after the use of costimulation blockade therapy to treat or prevent the autoimmune or inflammatory disease.

37. The method or costimulation blockade therapy for use according to any one of the preceding paragraphs, wherein determining the profile of B helper T cells in the sample is carried out prior to the onset of symptoms of the autoimmune or inflammatory disease.

38. A computer-readable medium comprising instructions that when executed cause one or more processors to perform the method of any one of paragraphs 1, 2, 4-13 or 29-37.

39. An apparatus comprising:
(a) profile determination circuitry to determine the profile of B helper T cells in a sample from a subject with an autoimmune or inflammatory disease; and
(b) subject identification circuitry to identify, based on the profile determination circuitry, a suitability of the subject for treatment with costimulation blockade therapy.

1. A method for identifying a subject with an autoimmune or inflammatory disease who is suitable for treatment with costimulation blockade therapy, the method comprising determining the profile of B helper T cells in a sample from the subject.

2. A method for predicting or determining whether a subject with an autoimmune or inflammatory disease will respond to treatment with costimulation blockade therapy, the method comprising determining the profile of B helper T cells in a sample from the subject.

3. The method according to claim 1 or 2, wherein the profile of B helper T cells is determined using at least one marker on CD4⁺ T cells selected from the group consisting of CXCR5, ICOS, PD-1, CD45RA, CD127 and/or CD25.

4. The method according to any one of the preceding claims, wherein the frequency of at least one B helper T cell phenotype is determined, optionally wherein the at least one B helper T cell phenotype is selected from the group consisting of ICOS -PD-1⁻ follicular helper T cells (Tfh), ICOS⁺ Tfh, CCR7-PD-1⁺ Tfh, CXCR5⁺ICOS⁺ T cells, CXCR5-ICOS⁺ T cells, ICOS⁺PD-1^{high} Tfh, ICOS-PD-1- memory T cells, ICOS-PD-1⁺ memory T cells and CXCR5⁺ naïve T cells.

5. The method according to claim 4, wherein the frequency of at least three B helper T cell phenotypes is determined.

6. The method according to claim 5, wherein the at least three B helper T cell phenotypes are ICOS ⁻PD-1⁻ Tfh, ICOS⁺ Tfh and CCR7-PD-1⁺ Tfh.

7. The method according to any one of the preceding claims, further wherein the frequency of at least one of naïve T cells and/or regulatory T cells (Treg) is determined in the sample from the subject.

8. The method according to any one of the preceding claims, wherein the profile of B helper T cells in the sample is compared to a reference frequency, wherein the reference frequency is selected from:
(a) a population of subjects who are non-responsive to the costimulation blockade therapy; and/or
(b) a population of subjects who are responsive to the costimulation blockade therapy.

9. The method according to claim 8, wherein the reference frequency is from a population of subjects who are non-responsive to the costimulation blockade therapy and wherein:
(a) a higher frequency of ICOS⁻PD-1⁻ Tfh;
(b) a lower frequency of ICOS⁺ Tfh;
(c) a lower frequency of CCR7-PD-1⁺ Tfh;
(d) a lower frequency of CXCR5⁺ICOS⁺ T cells;
(e) a lower frequency of CXCR5⁻ICOS⁺ T cells;
(f) a lower frequency of ICOS⁺PD-1^{high} Tfh;
(g) a higher frequency of ICOS⁻PD-1⁻ memory T cells;
(h) a higher frequency of ICOS⁻PD-1⁺ memory T cells;
(i) a lower frequency of CXCR5⁺ naïve T cells;
(j) a higher frequency of naïve T cells; and/or
(k) a higher frequency of Treg,
in comparison to a reference frequency is indicative of response to the treatment.

10. The method according to any one of the preceding claims, wherein the method comprises using at least one predictive modelling approach to identify the subject suitable for treatment with costimulation blockade therapy or to predict or determine whether the subject will respond to treatment with costimulation blockade therapy.

11. The method according to claim 10, wherein the at least one predictive modelling approach is gradient boosting, random forests, support vector machines and/or logistic regression.

12. The method according to claim 10 or claim 11, wherein populations of subjects grouped according to clinical response are used as inputs to the predictive modelling approach.

13. A costimulation blockade therapy for use in a method of treatment or prevention of an autoimmune or inflammatory disease in a subject, the method comprising:
(a) identifying or determining a subject with or at risk of developing an autoimmune or inflammatory disease who is suitable for treatment with costimulation blockade therapy by the method according to any one of claims 1-12; and
(b) treating the subject with costimulation blockade therapy.

14. A costimulation blockade therapy for use in treating or preventing an autoimmune or inflammatory disease in a subject, wherein the subject has been identified as suitable for treatment with the costimulation blockade therapy by determining the profile of B helper T cells in a sample from the subject.

15. The costimulation blockade therapy for use according to claim 14, wherein the profile of B helper T cells is determined according to the method of any of claims 1 to 12.

16. The method or costimulation blockade therapy for use according to any one of the preceding claims, wherein the autoimmune or inflammatory disease is selected from the group consisting of type 1 diabetes, rheumatoid arthritis, psoriatic arthritis, juvenile idiopathic arthritis, Sjogren’s syndrome, Graves’s Disease, Myasthenia Gravis, inflammatory vascular diseases, glomerulonephritis, diabetic nephropathy and systemic lupus erythematosus including systemic lupus erythematosus arthritis.

17. The method or costimulation blockade therapy for use according to any one of the preceding claims, wherein the autoimmune disease is type 1 diabetes.

18. The method or costimulation blockade therapy for use according to any one of the preceding claims, wherein the sample is a blood sample.

19. The method or costimulation blockade therapy for use according to any one of the preceding claims, wherein the costimulation blockade therapy is CD28 costimulation blockade therapy.

20. The method or costimulation blockade therapy for use according to claim 19, wherein the CD28 costimulation blockade therapy is selected from the group consisting of a CTLA-4-Ig fusion protein, such as Abatacept, Belatacept and MED15265; an anti-CD28 antagonist antibody, such as lulizumab; and FR104.

21. The method or costimulation blockade therapy for use according to any one of the preceding claims, wherein the profile of B helper T cells is determined by flow cytometry.

22. The method or costimulation blockade therapy for use according to any one of the preceding claims, wherein determining the profile of B helper T cells in the sample is carried out:
(a) prior to the onset of symptoms of the autoimmune or inflammatory disease;
(b) while the subject is showing symptoms of the autoimmune or inflammatory disease;
(c) prior to the use of costimulation blockade therapy to treat the autoimmune or inflammatory disease; and/or
(d) during and/or after the use of costimulation blockade therapy to treat the autoimmune or inflammatory disease.

23. A computer-readable medium comprising instructions that when executed cause one or more processors to perform the method of any one of claims 1 to 12.

24. An apparatus comprising:
(a) profile determination circuitry to determine the profile of B helper T cells in a sample from a subject with an autoimmune or inflammatory disease; and
(b) subject identification circuitry to identify, based on the profile determination circuitry, a suitability of the subject for treatment with costimulation blockade therapy.
